(19) United States
(12) Patent Application Publication
(10) Pub. No.: US 2001/0053849 A1
(43) Pub. Date: Dec. 20, 2001

(54) PLURAL BIOLOGICAL SAMPLE ARRAYS, AND PREPARATION AND USES THEREOF

(76) Inventors: MARY JEANNE KREEK, NEW YORK, NY (US); KARL STEVEN LAFORGE, NEW YORK, NY (US); RUDOLPH SPANGLER, NEW YORK, NY (US)

Correspondence Address: DAVID A JACKSON ESQ, KLAUBER & JACKSON, 411 HACKENSACK AVENUE, HACKENSACK, NJ, 07601

(21) Appl. No.: 09/334,113

(22) Filed: Jun. 16, 1999

(51) Int. Cl. C07H 21/00; C07K 17/00; C12Q 1/68; G01N 33/53; G01N 15/00

(52) U.S. Cl. 536/25.3; 435/6; 422/68.1; 435/7.1; 530/333

(57) ABSTRACT

The present invention relates to the high throughput analysis of polymorphisms of a family of genes associated with addiction and alcohol dependence. Included are probes prepared by a variety of techniques, a sample plate that may utilize DNA chip-type technology. The invention is adapted to identify both physiological and genetic conditions of subjects so tested, and should provide a rapid and inexpensive means for accomplishing the same.

### FIELD OF THE INVENTION

This invention relates to methods for concurrently performing multiple biological assays by means of gel pads or chips containing microarrays of biological material, and more particularly to the examination of particular genes associated with or affected by neurottransmitters. The invention further extends to the identification and consequent prognostication and implementation of corresponding therapy for conditions that cause genetic abnomalities or aberrations particularly those that result from excessive exposure to addictive agents and alcohol. The invention extends to the fields of chemistry, biology, medicine and diagnostics.

### BACKGROUND OF THE INVENTION

New technology, called VLSIPS™, has enabled the production of chips smaller than a thumbnail that contain hundreds of thousands or more of different molecular probes. These biological chips or arrays have probes arranged in arrays, each probe assigned a specific location. Biological chips have been produced in which each location has a scale of, for example, ten microns. The chips can be used to determine whether target molecules interact with any of the probes on the chip. After exposing the array to target molecules under selected test conditions, scanning devices can examine each location in the array and determine whether a target molecule has interacted with the probe at that location.

Biological chips or arrays are useful in a variety of screening techniques for obtaining information about either the probes or the target molecules. For example, a library of peptides can be used as probes to screen for drugs. The peptides can be exposed to a receptor, and those probes that bind to the receptor can be identified.

Arrays of nucleic acid probes can be used to extract sequence information from, for example, nucleic acid samples. The samples are exposed to the probes under conditions that allow hybridization. The arrays are then scanned to determine to which probes the sample molecules have hybridized. One can obtain sequence information by careful probe selection and using algorithms to compare patterns of hybridization and non-hybridization. This method is useful for sequencing nucleic acids, as well as sequence checking. For example, the method is useful in diagnostic screening for genetic diseases or for the presence and/or identity of a particular pathogen or a strain of pathogen.

Of particular interest herein are the abnormalities or polymorphisms that develop in genes that code for proteins the expression of which is known to be affected by narcotics such as opiates, cocaine or alcohol. Drug addiction continues to be a major medical and social problem. It is estimated that one million or more persons in the United States are currently addicted to heroin, with millions more worldwide. Cocaine addiction and alcohol dependence are frequent co-morbid conditions in heroin addicts in addition to being major primary addictions. Many studies over the past thirty years have shown that these drugs disrupt physiologic systems, and that these disruptions may contribute to drug addiction and alcohol dependence and to relapse to drug or alcohol abuse following withdrawal and abstinence. Clinical observations suggest that individuals differ in their response to heroin, cocaine, and alcohol; however, little is known about specific underlying hereditary genetic factors which might influence individual susceptibility to the addictive properties of these substances. Recent studies in genetic epidemiology provide evidence for heritable contributions to drug addiction in general and also heroin addiction specifically. A heritable basis for alcohol dependence has long been established. Furthermore, there is evidence that both common and distinct genetic factors underlie some of the susceptibility for these addictive diseases. Clearly an interaction of both environmental and genetic factors play a role in the addictions.

It is hypothesized that polymorphism exists in genes involved in the biological responses to heroin, cocaine, and alcohol, and that some of these polymorphisms will result in variant forms of the proteins they encode. Other polymorphisms which do not result in amino acid changes will be useful in association and linkage studies and also in genome scans. Those polymorphisms which do result in changes in amino acid structure should be studied for function, as it is further hypothesized that some of the individual variations in responses to acute or chronic exposure to, or withdrawal from, heroin, cocaine, and alcohol may be mediated, in part, by the variant forms of these proteins. In addition, it is believed that other genes may be involved in the development and persistence of addiction and in relapse, and that these genes may be identified by a genome scan of affected sib pairs rigorously characterized with respect to the addictive diseases and related co-morbid conditions. Thus, the genes of interest herein would desirably be studied with the assistance of the high throughput capabilities of contemporary biological array technology.

With respect to the preparation of biological arrays, devices and corresponding methods have been developed that are capable of handling multiple samples simultaneously. For example, U.S. Pat. No. 5,545,531 to Rava et al. discloses a device that can process 96 wells, each having probe arrays that, in turn, can define as many as 1,000,000 probes. Also, U.S. Pat. No. 5,858,661 to Shiloh illustrates the full exposition of a particular gene, and includes DNA chip analysis as a means of exploiting the information regarding the gene for patient analyis. To date however, the particular family of genes of interest herein and the manner in which they would be disposed on such an array and studied has not been considered or addressed, and it is to the achievement of this and related objectives that the present invention is directed. Naturally, the ability to conduct such studies in a thorough and rapid manner is highly desirable.

The citation of any reference herein should not be construed as an admission that such reference is available as “Prior Art” to the instant application.

### SUMMARY OF THE INVENTION

The present invention provides a novel means of studying genes of interest and relevance to a variety of neurological disorders and dysfunctions, and particularly, those genes affected by exposure to agents of addiction and alcohol dependence. Specifically, the invention extends to a device providing a biological array on which there are disposed a plurality of DNA and RNA sequences corresponding to the genes of interest. This array provides a multifunction analytical capability, as it facilitates the study of RNA abnormalities or polymorphisms, and particularly single nucleotide polymorphisms (SNPs), will yield quantitative information as to the physiological and/or pathological condition of the test subject, while the analysis of the DNA of the subject will provide information regarding subject genotype and corresponding genetic predisposition.

More particularly, the biological arrays useful herein include those arrays prepared by the solid phase techniques as disclosed in Rava et al. supra., as well as the use of polymeric gel affixation of multiple oligonucleotide strands to e.g. a glass plate, as disclosed by Yershov et al. (1996) Proc. Natl. Acad. Sci. USA 93:4913-4918, the disclosures of which are incorporated herein by reference in their entireties. Other means and techniques for disposing plural biological materials on a solid surface are contemplated herein and considered to be a part hereof.

The invention relates to the study of both RNA and DNA to discover and analyze the significance of polymorphic changes, extending to single nucleotide polymorphisms (SNPs) of a large family of neurotransmitter factors. The family of materials and genes intended herein, includes those genes involved with the following exemplary physiological and pathological states and conditions: addiction; response to pain; stress; gastrointestinal function; immune function; reproductive function; and signal transduction.

Particular genes of interest include the opioid system, such as, the kappa opioid receptor and preprodynorphin, the mu receptor, the delta receptor, preproenkephalin, the opioid-like receptor (OLR1) and orphanin FQ/ (nociceptin), corticotrophin releasing factor and the corticotrophin releasing factor receptor type I, preproopiomelanocortin, and related peptide ligands; the dopaminergic system, including Dopaminergic receptors D1-D5, the dopamine transporter; the serotonin system, including serotonin and melatonin, their particular metabolic and synthetic interrelation, and 15 serotonin receptors, and the serotonin transporter; the norepinephrin receptor, and related molecules, and signal transducers, such as adenylyl cyclase and DARPP-32 the activity cycle of the latter which is controlled by interaction with dopamine, dopamine D1 and D2 receptors, and calcineurin. DARPP-32 is thought to play a role in diseases such as schizophrenia, Parkinson's disease, Tourette's syndrome, drug abuse and attention deficit disorder. In addition, the present invention will lead to and thereby comprehends within its scope, methods for identifying agents that can be used in such treatment.

The studies in accordance with the invention are performed using both traditional and novel approaches for DNA sequencing and identification of SNPs and other polymorphisms. Distribution of allele and genotype frequencies is to be defined with respect to ethnicity; association of specific alleles and genotypes with opiate addiction, and also with cocaine addiction and alcohol dependency, may be studied. Classical case-control and sib pair association and linkage disequilibrium methods are used.

The present invention may utilize a biological chip plate comprising a plurality of test wells. Each test well defines a space for the introduction of a sample and contains a biological array. The array is formed on a surface of the substrate, with the probes exposed to the space. A fluid handling device manipulates the plates to perform steps to carry out reactions between the target molecules in samples and the probes in a plurality of test wells. The biological chip plate is then interrogated by a biological chip plate reader to detect any reactions between target molecules and probes in a plurality of the test wells, thereby generating results of the assay. In a further embodiment of the invention, the method may also include processing the results of the assay with a computer. Such analysis would be useful e.g. when sequencing a gene by a method that uses an algorithm to process the results of many hybridization assays to provide the nucleotide sequence of the gene.

The methods of the invention can involve the binding of tagged target molecules to the probes. The tags can be, for example, fluorescent markers, chemiluminescent markers, light scattering markers or radioactive markers. In certain embodiments, the probes are nucleic acids, such as DNA or RNA molecules. The methods can be used to detect or identify polymorphisms resulting from e.g. a pathogenic organism, or from the excessive exposure to damaging agents such as opiates and alcohol, or to detect a human gene variant, such a the gene for a genetic disease such as cystic fibrosis, diabetes, muscular dystrophy or the predisposition to certain neurological disorders.

This invention also provides systems for performing the methods of this invention. In an exemplary embodiment, the systems include a biological chip plate; a fluid handling device that automatically performs steps to carry out assays on samples introduced into a plurality of the test wells; a biological chip plate reader that determines in a plurality of the test wells the results of the assay and, optionally, a computer comprising a program for processing the results. The fluid handling device and plate reader can have a heater/cooler controlled by a thermostat for controlling the temperature of the samples in the test wells and robotically controlled pipets for adding or removing fluids from the test wells at predetermined times.

In certain embodiments, the probes are attached by light-directed probe synthesis. The biological chip plates can have 96 wells arranged in 8 rows and 12 columns, such as a standard microtiter plate. The probe arrays can each have at least about 100, 1000, 100,000 or 1,000,000 addressable features (e.g., probes). A variety of probes can be used on the plates, including, for example, various polymers such as peptides or nucleic acids.

The plates can have wells in which the probe array in each test well is the same. Alternatively, when each of several samples are to be subjected to several tests, each row can have the same probe array and each column can have a different array. Alternatively, all the wells can have different arrays.

Several methods of making biological chip plates are contemplated. In a method presented herein by way of non-limiting example, a wafer and a body are provided. The wafer includes a substrate and a surface to which is attached a plurality of arrays of probes. The body has a plurality of channels. The body is attached to the surface of the wafer whereby the channels each cover an array of probes and the wafer closes one end of a plurality of the channels, thereby forming test wells defining spaces for receiving samples. In a second method, a body having a plurality of wells defining spaces is provided and biological chips are provided. The pads or chips are attached to the wells so that the probe arrays are exposed to the space. Another embodiment involves providing a wafer having a plurality of probe arrays; and applying a material resistant to the flow of a liquid sample so as to surround the probe arrays, thereby creating test wells.

This invention may utilize a wafer for making a biological sample plate. The wafer has a substrate and a surface to which are attached a plurality of probe arrays. The probe arrays are arranged on the wafer surface in rows and columns, wherein the probe arrays in each row are the same and the probe arrays in each column are different.

Accordingly, it is a principal object of the present invention to provide a method and corresponding devices for the concurrent study and analysis of genetic material of subjects suspected of having genetic or pathological injury resulting from excessive exposure to addictive substances or alcohol.

It is a further object of the present invention to provide a method as aforesaid that examines both RNA and DNA to identify any polymorphisms including single nucleotide polymorphisms.

It is a further object of the present invention to provide a method as aforesaid that is a method for diagnosing pathology and/or identifying genetic predisposition of a test subject toward a particular deleterious condition.

It is a yet further object of the present invention to provide a method as aforesaid that may be used to identify new therapeutic agents by virtue of their ability to modulate the incidence of such polymorphisms.

It is a still further object of the invention to prepare and use a biological array that includes all of the various genes associated with neurotransmitter molecules, and particularly those associated with addiction and alcohol abuse, for the efficient and thorough study of patient tissue and genetic material.

These and other aspects of the present invention will be better appreciated by reference to the following drawings and Detailed Description.

### BRIEF DESCRIPTION OF THE DRAWINGS

FIG. 1

### DETAILED DESCRIPTION OF THE INVENTION

The present invention has as among its objects, the development and use of a facile method and corresponding materials for the study of plural genes and other factors believed to be affected by addictive agents and alcohol. Particularly, the invention contemplates and covers the identification of polymorphism in DNA and/or RNA from or associated with these genes or agents, and the corresponding pathological and diagnostic and therapeutic information regarding the genes of interest. The genes in object are those associated with addiction and dependencies such as alcohol dependency.

Accordingly, the present invention proposes to study the entire family of neurotransmitter genes and particularly, those associated with addiction and dependency, by the disposition of plural DNA and/or RNA fragments or probes in multiple arrays for high throughput screening. As statued earlier and as contemplated herein, the devices that may be used include the multiple arrays known as DNA chips or the like, as set forth in U.S. Patent to Rava et al., discussed earlier and incorporated herein by reference.

Thus, to the extent that the following terms are used herein, they are intended to have the following general meanings:

Complementary: Refers to the topological compatibility or matching together of interacting surfaces of a probe molecule and its target. Thus, the target and its probe can be described as complementary, and furthermore, the contact surface characteristics are complementary to each other.

Probe: A probe is a surface-immobilized molecule that can be recognized by a particular target. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies. Particular probes of interest herein include DNA and RNA derived from genes affected by addictive agents and alcohol, such as those listed above and herein.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term “targets” is used herein, no difference in meaning is intended. A “Probe Target Pair” is formed when two macromolecules have combined through molecular recognition to form a complex.

Array: A collection of probes, at least two of which are different, arranged in a spacially defined and physically addressable manner.

Biological Chip: A substrate having a surface to which one or more arrays of probes is attached. The substrate can be, merely by way of example, silicon or glass and can have the thickness of a glass microscope slide or a glass cover slip. Substrates that are transparent to light are useful when the method of performing an assay on the chip involves optical detection. As used herein, the term also refers to a probe array and the substrate to which it is attached that form part of a wafer.

Wafer: A substrate having a surface to which a plurality of probe arrays are attached. On a wafer, the arrays are physically separated by a distance of at least about a millimeter, so that individual chips can be made by dicing a wafer or otherwise physically separating the array into units having a probe array.

Biological Chip Plate: A device having an array of biological chips in which the probe array of each chip is separated from the probe array of other chips by a physical barrier resistant to the passage of liquids and forming an area or space, referred to as a “test well,” capable of containing liquids in contact with the probe array.

The general class of genes of interest may be identified as neurological markers, and particularly, neurotransmitters. Ligand-gated ion channels represent a large, evolutionarily related group of intrinsic membrane proteins that form multisubunit complexes and transduce the binding of small agonists into transient openings of ion channels. Neurotransmitters bind to these channels externally, causing a change in their conformation, allowing ions to cross the membrane and thereby alter the membrane potential. The receptors which comprise these channels have an enzyme-like specificity for particular ligands (the neurotransmitters) and are characterized by their ion selectivities, including permeability to Na+, K+, Cl−, etc. Recognized neurotransmitters include acetylcholine, dopamine, serotonin, epinephrine, gamma-aminobutyrate (GABA), glutamate and glycine, each recognized by distinct receptors. The super-family of ligand-gated channels includes the nicotinic acetylcholine receptor (nAChR), the serotonin receptor, the GABA receptor, and glutamate receptors.

Neurotransmitters are synthesized in brain neurons and stored in vesicles. Upon a nerve impulse, a neurotransmitter is released into the synaptic cleft, where it interacts with various postsynaptic receptors. The actions of neurotransmitters, such as acetylcholine and serotonin, are terminated by three major mechanisms: diffusion; metabolism; and uptake back into the synaptic cleft through the actions of membrane transporter systems. Thus, the actions of any such neurotransmitter can be theoretically modulated by: agents that stimulate or inhibit its biosynthesis; agents that block its storage; agents that stimulate or inhibit its release; agents that mimic or inhibit its actions at its various postsynaptic receptors; agents that inhibit its uptake back into the nerve terminal; and agents that affect its metabolism.

The acetylcholine receptor (AChR) is divided into two main types, muscarinic and nicotinic, based on the fact that the two poisons nicotine (from tobacco), and muscarine (from mushrooms) mimic the effect of acetylcholine on different types of receptors. The muscarinic ACHR is found on smooth muscle, cardiac muscle, endocrine glands and the central nervous system (CNS). The nicotinic AChR (nAChR) is located on skeletal muscle, ganglia and the CNS, mediating synaptic transmission at the neuromuscular junction, in peripheral autonomic ganglia, and in the CNS.

Nicotinic acetylcholine receptors are glycosylated multisubunit pentamers. Six different types of subunit have been identified—alpha, beta, gamma, sigma, delta and epsilon- each of molecular weight 40-60 kDa. The pentamer is made up of different combinations of the subunits. The five subunits form a ring which spans the plasmamembrane of the postsynaptic cell, creating a channel. Within each subunit type, distinct subtypes have been identified, including multiple alpha subunits (α1-α9) and beta subunits (β2-β4) with related but unique sequences (Role and Berg (1996) Neuron 16, 1077-1085). The binding of acetylcholine or nicotine to the alpha subunit of the receptor induces a conformational change which allows the influx of sodium and calcium into the cell. The synaptic action of acetylcholine on the receptor is terminated by enzymatic cleavage by acetylcholinesterase.

CNS therapeutic applications for the acetylcholine receptors include cholinometic approaches in the treatment of Alzheimer's disease and anticholinergic drugs in the treatment of Parkinson's disease. Nicotinic cholinoceptive dysfunction associated with cognitive impairment is a leading neurochemical feature of the senile dementia of the Alzheimer type. For this reason, nicotinic acetylcholine receptors have attracted considerable interest as potential therapeutic targets in Alzheimer's disease. Nicotinic acetylcholine receptors have also been implicated as potential therapeutic targets in other memory, learning and cognitive disorders and deficits, including Lewy Body dementia and attention deficit disorder. In addition, the alpha subunit of nAChR has been recognized as playing an important role in the etiology of congenital myasthenia syndromes and stimulates T cells in patients with auto-immune mediated myasthenia gravis (Croxen, R. et al., (1997) Hum Mol Genet 6, 767-774; Sine, S. M. et al., (1995) Neuron 15, 229-239; Katz-Levy, Y. et al., (1998) J. Neuroimmunol 85, 78-86).

Located primarily in peripheral and central neurons, serotonin (5-hydroxytryptamine, 5-HT) receptors appear to be involved in the depolarization of peripheral neurons, pain, and the emesis reflex. Potential use of agents acting at this site include migraine, anxiety, substance abuse, and cognitive and psychotic disorders. There are at least four populations of receptors for serotonin: 5-HT1, 5-HT2, 5-HT3, and 5-HT4. Recent cloning studies suggest the existence of 5-HT5, 5-HT6, and 5-HT7 subtypes as well. In addition at least five distinct subtypes of the 5-HT2 and three subtypes of the 5-HT3 receptors exist. Largely due to the complexity of these multiple subtypes, the physiological function of each receptor subtype has not been fully established. With the exception of the 5-HT3 receptor, which is a ligand-gated ion channel related to NMDA, GABA and nicotinic receptors, all of the 5-HT receptor subtypes belong to the group of G-protein linked receptors.

Serotonin is implicated in the etiology or treatment of various disorders, including anxiety, depression, obsessive-compulsive disorder, schizophrenia, stroke, obesity, pain, hypertension, vascular disorders, migraine, and nausea. 5-HT is synthesized in situ from tryptophan through the actions of the enzymes tryptophan hydroxylase and aromatic L-amino acid decarboxylase. Both dietary and endogenous 5-HT are rapidly metabolized and inactivated by monoamine oxidase and aldehyde dehydrogenase to the major metabolite, 5-hydroxyindoleacetic acid (5-HIAA). The major mechanism by which the action of serotonin is terminated is by uptake through presynaptic membranes. After 5-HT acts on its various postsynaptic receptors, it is removed from the synaptic cleft back into the nerve terminal through an uptake mechanism involving a specific membrane transporter in a manner similar to that of other biogenic amines. Agents that selectively inhibit this uptake increase the concentration of 5-HT at the postsynaptic receptors and have been found to be quite useful in treating various psychiatric disorders, particularly depression. Selective 5-HT reuptake inhibitors (SSRIs) have been investigated as potential antidepressants with the anticipation that these agents would possess fewer side effects, such as anticholinergic actions and cardiotoxicity, and would be less likely to cause sedation and weight gain.

Three selective 5-HT uptake inhibitors, have more recently been introduced on the U.S. market, Fluoxetine (Prozac), sertraline (Zoloft), and paroxetine (Paxil) and have gained immediate acceptance, each listed among the top 200 prescription drugs.

In addition to treating depression, several other potential therapeutic applications for SSRIs have been investigated. They include treatment of Alzheimer's disease; modulation of aggressive behavior; treatment of premenstrual syndrome, diabetic neuropathy, and chronic pain; and suppression of alcohol intake. Also significant is the observation that 5-HT reduces food consumption by increasing meal-induced satiety and reducing hunger, thus, there is interest in the possible use of SSRIs in the treatment of obesity.

5-HT3 receptors have been proposed to play a major role in the physiology of emesis. These receptors are found in high concentrations peripherally in the gut and centrally in the cortical and limbic regions and in or near the chemoreceptor trigger zone, and have been implicated in the vomiting reflex induced by serotonin as a result of chemotherapy. Two 5-HT3 receptor antagonists, ondansetron (zofran) and granisetron (Kytril), have been marketed to treat nausea associated with radiation and chemotherapy in cancer patients.

Several family, twin, and adoption studies provide evidence for heritable contributions to drug and alcohol dependency, although little is known about specific underlying hereditary factors which might influence individual susceptibility to the addictive properties of these substances [5-9] Recent familial and twin studies have reported that both common and distinct heritable factors account for the genetic variance in the susceptibility to the separate addictive diseases, i.e. that both shared and independent causative factors contribute to the development of each separate type of substance dependence [9-12]. Moreover, in a study of 3372 male twin pairs, Tsuang and colleagues [9,10] found that heroin abuse had the largest amount of unique genetic variance (38%) and the least amount of shared genetic variance (16%) of any of the other abused drugs studied (marijuana, stimulants, sedatives, psychedelics).

Animal studies also provide evidence for a genetic contribution to the addictive diseases. Different strains of rhodents have been shown to have differences in their responses to opioids, cocaine and alcohol in models which study self-administration, reinforcement, and tolerance, each of which may have potential implications for the susceptibility to develop drug addiction in humans. [e.g. 13-17].

Many studies over the past thirty years have shown that opioids, cocaine and alcohol disrupt physiologic systems, and that these disruptions may contribute to drug addiction and alcohol dependence and to relapse to drug or alcohol abuse following withdrawal and abstinence. It is hypothesized herein that polymorphism exists in genes involved in the biological responses to heroin, cocaine, and alcohol, and that some of these polymorphisms will result in variant forms of the proteins they encode. Further, some of the individual variations in responses to acute or chronic exposure to, or withdrawal from, heroin, cocaine, and alcohol may be mediated, in part, by variant allelic forms of these genes. Moreover, other heretofore undefined genes may be involved in the development and persistence of addiction and in relapse, and that these genes may be identified by using genomic scans of sib pairs rigorously characterized with respect to the addictive diseases and related comorbid conditions.

From the foregoing, it can be appreciated that a broad physiological and pathological range and effect is commanded by these molecules. As noted earlier, the present invention is applicable to the synthesis and study of any of the molecules included within this class, however, focuses its primary attention on the molecules referred to earlier and discussed in detail below.

Accordingly and as stated above, the genes in question are found among the following neurotransmitters: the opioid system, such as, the kappa opioid receptor and preprodynorphin, the mu receptor, the delta receptor, preproenkephalin, the opioid-like receptor (OLR1) and orphanin FQ/ (nociceptin), corticotrophin releasing factor and the corticotrophin releasing factor receptor type I, preproopiomelanocortin, and related peptide ligands; the dopaminergic system, including Dopaminergic receptors D1-D5, the dopamine transporter; the serotonin system, including serotonin and melatonin, their particular metabolic and synthetic interrelation, and 15 serotonin receptors, and the serotonin transporter; the norepinephrin receptor, and related molecules, and signal transducers, such as adenylyl cyclase and DARPP-32.

More particularly, the following genes will be studied with a view to the examination of particular polymorphisms, as follows:

The kappa opioid receptor gene (KOR). The coding region of the KOR gene has been shown to be dispersed in three exons of 264, 352 and 533 bp in length [18,19]. The intron sequences flanking the 3′ end of exon 2 is available in GenBank (Accession #U16860). The rest of the intron sequences flanking exon 2 and exon 3 have been examined, and have provided the information necessary to design primers for PCR amplification of exons 2 and 3. The sequences flanking exon 1 may be obtained by inverse PCR. Nested primers will be used for manual and automated sequencing of exon 1, 2 and 3.

The preprodynorphin gene (ppDyn). DNA of this gene may be analyzed for polymorphisms in and around exons 1, 3 and 4 of the ppDyn gene (exon 2 contains only 5′ untranslated sequence). Translation starts in exon 3 and ends in exon 4, which encodes the opioid peptides. The nucleotide sequence of the exons and flanking intron sequences are available in GenBank (accession #X00175, X0177). Primers completely flanking exons 1 and 3 may be used for determination of sequence in those exons, and primers downstream of the exon 4 border together with primers in the 3 untranslated region of exon 4 may be used for determination of sequence in exon 4.

The opioid receptor-like receptor (ORL 1). The primary structure of the gene has been reported [21]. The coding region of the receptor is interrupted by a single short 120 bp intron. The published sequences flanking the coding regions of ORL1 will be used to design PCR and sequencing primers.

The orphanin FQ gene (prepronociceptin). The orphanin FQ gene is composed of 4 exons [22]. Translation starts in exon 2 and the biologically active heptadecapeptide is encoded in exon 3. The sequences flanking exons 2 and 3 will be used for PCR and sequencing primer design.

The preproenkephalin gene (ppENK). The ppENK gene and cDNA sequences have been published [23,24]. The ppENK gene consists of 3 exons. The opioid peptides are located in exon 3. Primers completely flanking exon 2 may be used for determination of sequence in that exon, and primers downstream of the exon 3 border together with primers in the 3′ untranslated region of exon 3 may be used for determination of sequence in exon 3.

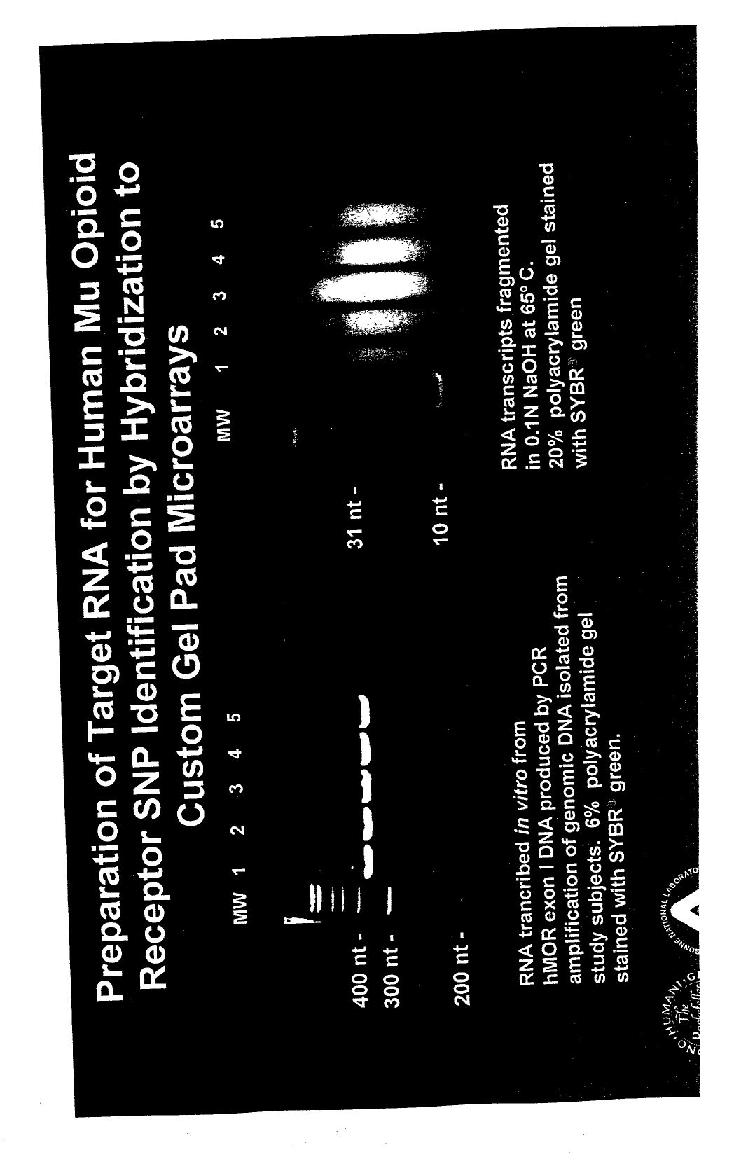

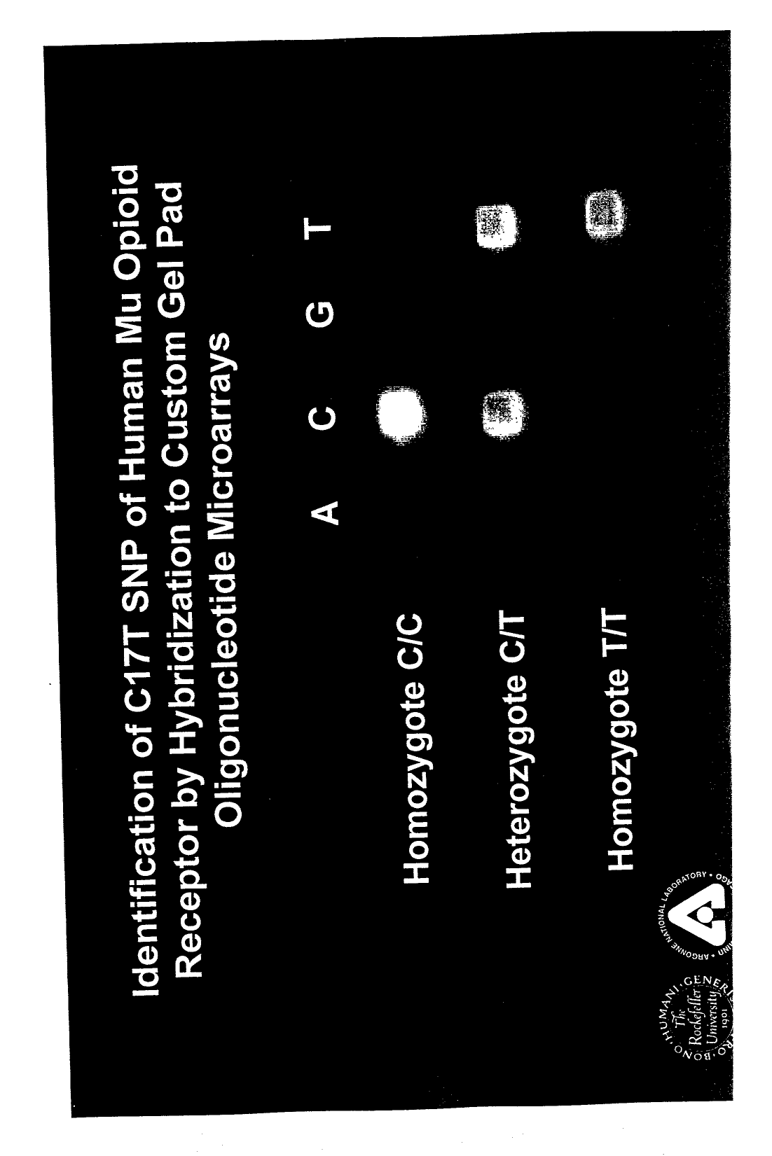

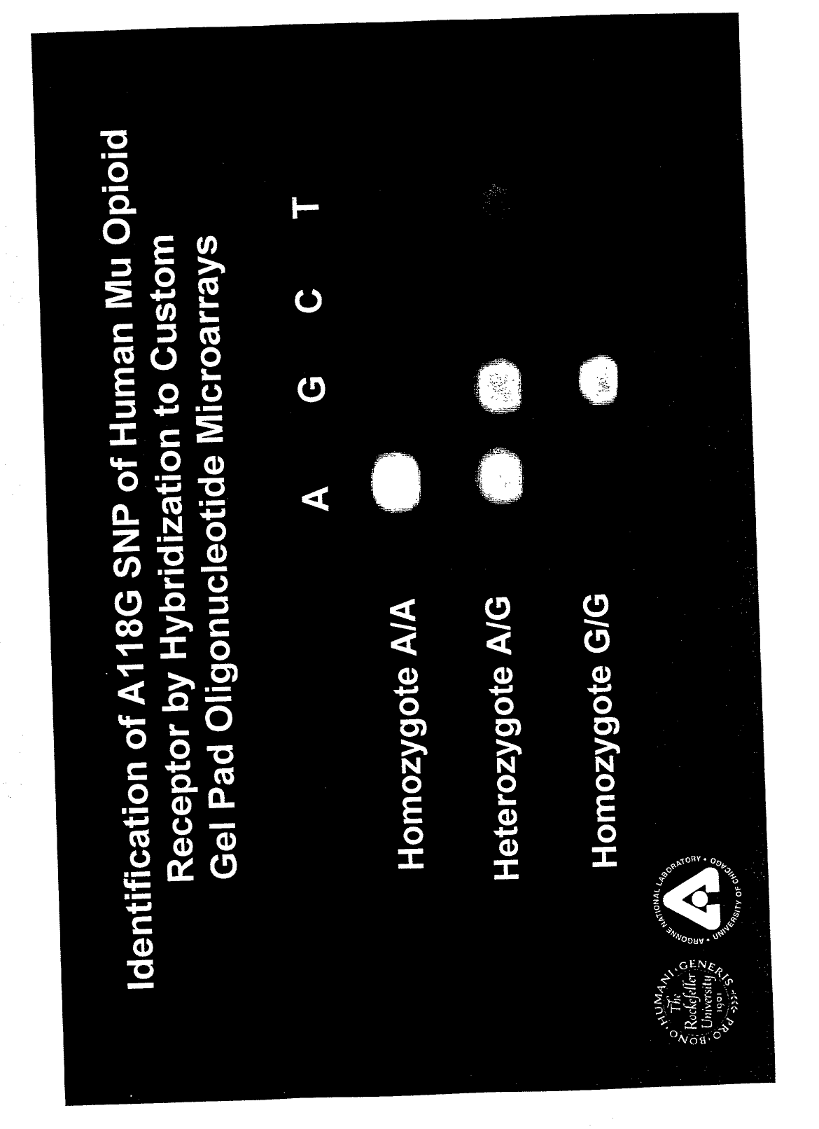

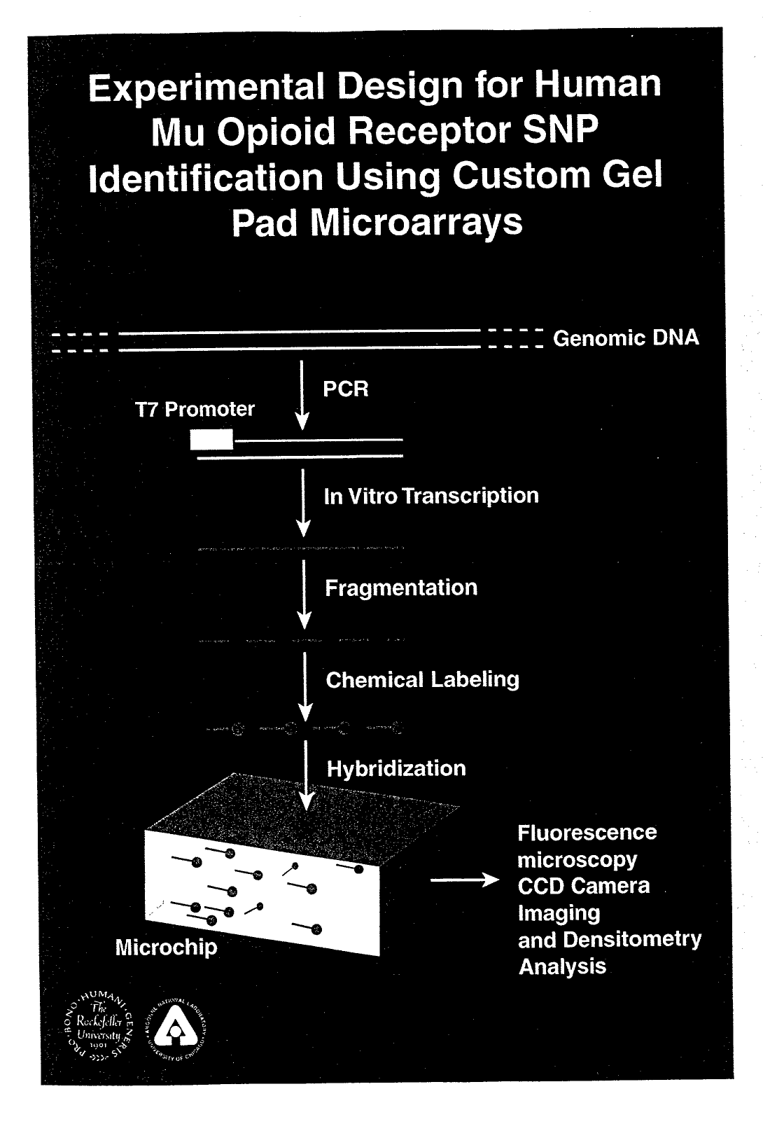

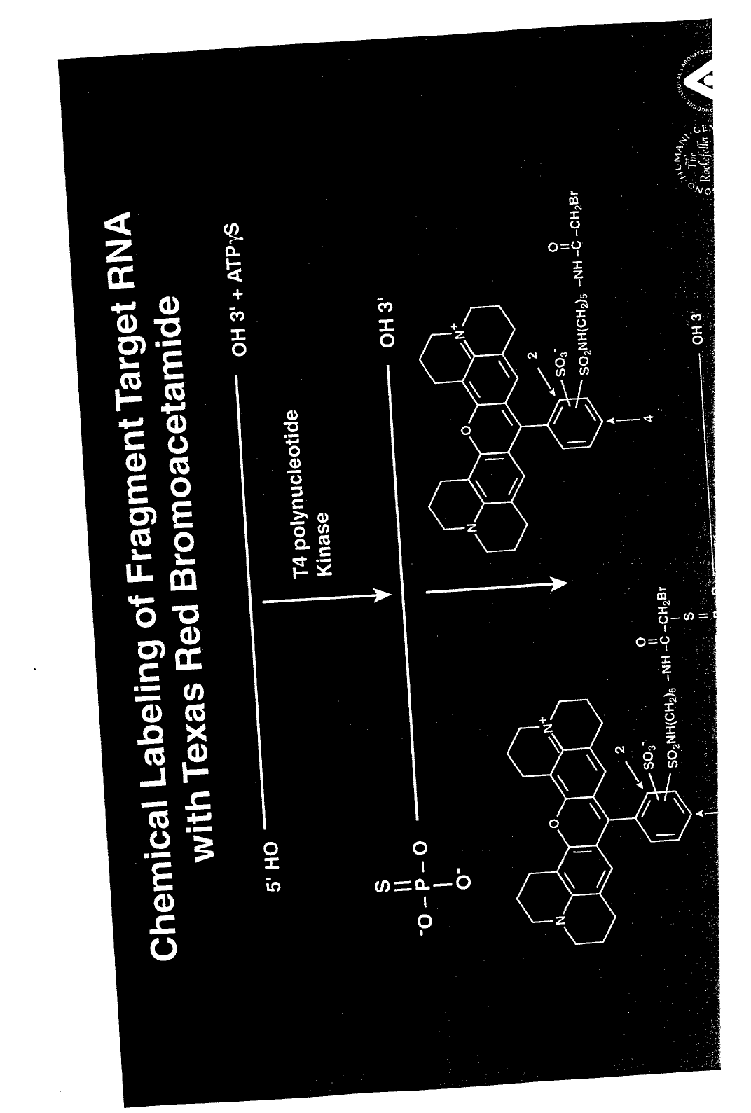

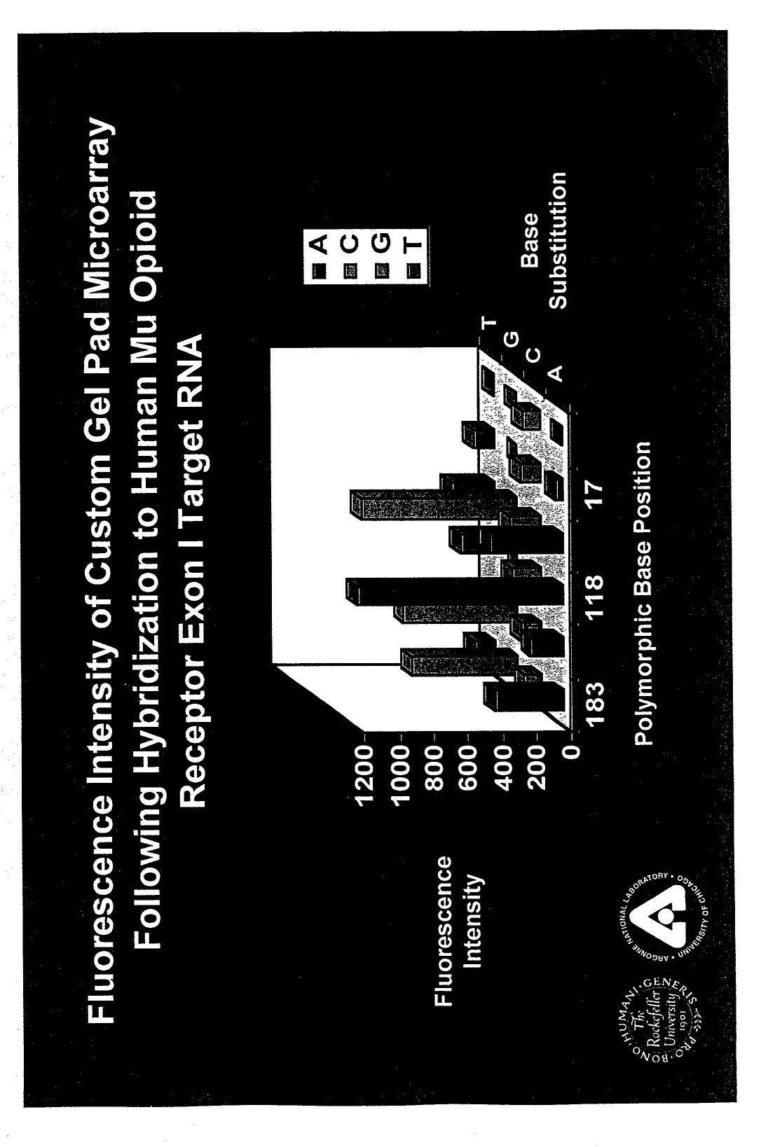

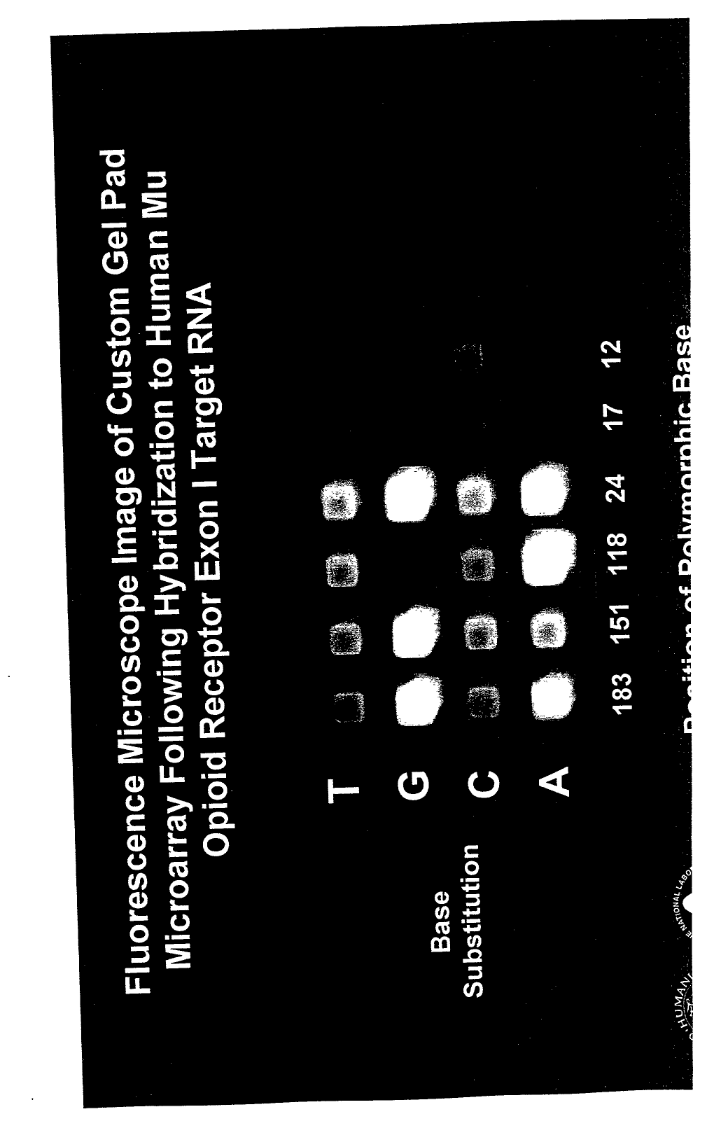

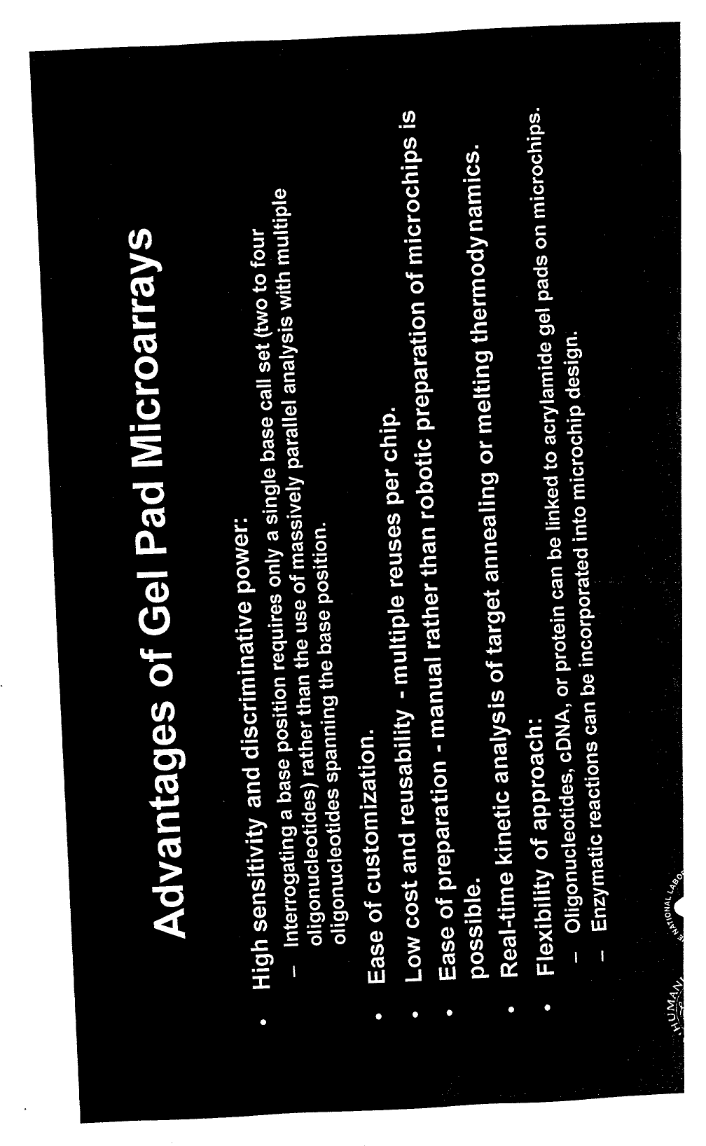

The corticotropin releasing factor gene (CRF). The CRF gene structure has been published [25]. The CRF gene consists of two exons, with all the uninterrupted sequence of the CRF precursor (196 amino acid) in exon 2. A primer flanking exon 2 upstream of the intron/exon border may be used, and the same primer in the 3′ untranslated region used to generate the fragment shown in FIG. 1, lane d, for determination of significant sequence from the CRF gene.

The corticotropin releasing factor receptor, type1 gene (CRF-R1). A cDNA sequence encoding the 415 amino acid human CRF-R1 protein has been reported [26,27]. The genomic structure is apparently not yet publicly known. However, there is an apparently alternatively spliced form of the CRFR1 mRNA in which 29 amino acids are inserted into the first intracellular loop. The site of the insertion indicates the position of a putative intron. In order to obtain the intron sequences, we will use PCR amplification of human genomic DNA with primers flanking the insert in CRF-R1. Sequencing of this putative intron region will enable us to design PCR and sequencing primers for the coding region of CRF-R1. To define the intron/exon structure of the rest of the gene overlapping sets of primer pairs will be designed which amplify short sections(˜200 bp) of the coding region. Genomic DNA will be amplified using these primer sets and products will be analysed for amplicons of the predicted length. If longer fragments than expected are produced, or if intron sequences are present that are too long to successfully amplify, this will indicate the approximate position of introns. Exact intron/exon boundaries will then be determined by inverse PCR as described [171].

The preproopiomelanocortin gene (POMC). The gene and cDNA structure of POMC have been reported [28-30]. The POMC gene consists of 3 exons. The coding regions for the biologically active peptides, ACTH and beta-lipotropin, and their smaller derivatives, alpha-melanotropin, beta-melanotropin and beta-endorphin, are located in exon 3.

As stated earlier, this invention provides automated methods for concurrently processing multiple biological chip assays. Currently available methods utilize each biological chip assay individually. The methods of this invention allow many tests to be set up and processed together. Because they allow much higher throughput of test samples, these methods greatly improve the efficiency of performing assays on biological chips.

In the methods of this invention, a biological chip plate is provided having a plurality of test wells. Each test well includes a biological chip. Test samples, which may contain target molecules, are introduced into the test wells. A fluid handling device exposes the test wells to a chosen set of reaction conditions by, for example, adding or removing fluid from the wells, maintaining the liquid in the wells at predetermined temperatures, and agitating the wells as required, thereby performing the test. Then, a biological chip reader interrogates the probe arrays in the test wells, thereby obtaining the results of the tests. A computer having an appropriate program can further analyze the results from the tests.

Individual chips may have attached to them a plurality of probes, the probes in turn prepared by the following exemplary protocol. Thus, sequences flanking coding regions of human receptor and prepropeptide genes may be used to design PCR primers for use in the amplification. Optimal forward and reverse primers are selected with the aid of the primer analysis software, Oligo 4.1 (National Biosciences, MN). We will use step-down PCR [170], which will add specificity during those cycles above the melting temperature (Tₘ) of an oligonucleotide duplex, as well as enhanced efficiency during those cycles below the Tₘ, to simultaneously increase both product yield and homogeneity. Preliminary optimization of annealing temperature and PCR cycling is performed using the Eppendorf Mastercycler Gradient. PCR amplification is carried out in 50 to 100 μl reactions with 200 ng genomic DNA, 20 pmol of each primer, 200 mM of each dNTP, 50 mM KCl, 10 mM Tris-HCl (pH 8.3), 1.5 mM MgCl₂, and 2.5 U Taq polymerase. Samples will be cycled 30 sec at 94° C., with annealing for 45 sec at a variable (step-down) or a fixed temperature, then elongation for 30 sec at 72° C., followed by a final elongation period of 5 min at 72° C. PCR products will be analyzed by electrophoresis in agarose gels and visualized by ethidium bromide staining. Single band PCR products will be purified by QIAquick PCR purification Kit (Qiagen); if there is more than one fragment, the correct fragment will be isolated from the gel and purified by QIAquick Gel Extraction Kit (Qiagen).

Further, an exemplary system includes a biological chip plate reader, a fluid handling device, a biological chip plate and, optionally, a computer. In operation, samples are placed in wells on the chip plate with fluid handling device. The plate optionally can be moved with a stage translation device. The reader is used to identify where targets in the wells have bound to complementary probes. The system operates under control of computer which may optionally interpret the results of the assay.

A. Biological Chip Plate Reader

In assays performed on biological chips, detectably labeled target molecules bind to probe molecules. Reading the results of an assay involves detecting a signal produced by the detectable label. Reading assays on a biological chip plate requires a biological chip reader. Accordingly, locations at which target(s) bind with complementary probes can be identified by detecting the location of the label. Through knowledge of the characteristics/sequence of the probe versus location, characteristics of the target can be determined. The nature of the biological chip reader depends upon the particular type of label attached to the target molecules.

The interaction between targets and probes can be characterized in terms of kinetics and thermodynamics. As such, it may be necessary to interrogate the array while in contact with a solution of labeled targets. In such systems, the detection system must be extremely selective, with the capacity to discriminate between surface-bound and solution-born targets. Also, in order to perform a quantitative analysis, the high-density of the probe sequences requires the system to have the capacity to distinguish between each feature site. The system also should have sensitivity to low signal and a large dynamic range.

In one embodiment, the chip plate reader includes a confocal detection device having a monochromatic or polychromatic light source, a focusing system for directing an excitation light from the light source to the substrate, a temperature controller for controlling the substrate temperature during a reaction, and a detector for detecting fluorescence emitted by the targets in response to the excitation light. The detector for detecting the fluorescent emissions from the substrate, in some embodiments, includes a photomultiplier tube. The location to which light is directed may be controlled by, for example, an x-y-z translation table. Translation of the x-y-z table, temperature control, and data collection are managed and recorded by an appropriately programmed digital computer.

FIG. 2 of U.S. Pat. No. 5,545,531, illustrates a reader according to one specific embodiment. The chip plate reader comprises a body 200 for immobilizing the biological chip plate. Excitation radiation, from an excitation source 210 having a first wavelength, passes through excitation optics 220 from below the array. The light passes through the chip plate since it is transparent to at least this wavelength of light. The excitation radiation excites a region of a probe array on the biological chip plate 230. In response, labeled material on the sample emits radiation which has a wavelength that is different from the excitation wavelength. Collection optics 240, also below the array, then collect the emission from the sample and image it onto a detector 250, which can house a CCD array, as described below. The detector generates a signal proportional to the amount of radiation sensed thereon. The signals can be assembled to represent an image associated with the plurality of regions from which the emission originated.

According to one embodiment, a multi-axis translation stage 260 moves the biological chip plate to position different wells to be scanned, and to allow different probe portions of a probe array to be interrogated. As a result, a 2-dimensional image of the probe arrays in each well is obtained.

The biological chip reader can include auto-focusing feature to maintain the sample in the focal plane of the excitation light throughout the scanning process. Further, a temperature controller may be employed to maintain the sample at a specific temperature while it is being scanned. The multi-axis translation stage, temperature controller, auto-focusing feature, and electronics associated with imaging and data collection are managed by an appropriately programmed digital computer 270.

In one embodiment, a beam is focused onto a spot of about 2 μm in diameter on the surface of the plate using, for example, the objective lens of a microscope or other optical means to control beam diameter.

In another embodiment, fluorescent probes are employed in combination with CCD imaging systems. In many commercially available microplate readers, typically the light source is placed above a well, and a photodiode detector is below the well. In the present invention, the light source can be replaced with a higher power lamp or laser. In one embodiment, the standard absorption geometry is used, but the photodiode detector is replaced with a CCD camera and imaging optics to allow rapid imaging of the well. A series of Raman holographic or notch filters can be used in the optical path to eliminate the excitation light while allowing the emission to pass to the detector. In a variation of this method, a fiber optic imaging bundle is utilized to bring the light to the CCD detector. In another embodiment, the laser is placed below the biological chip plate and light directed through the transparent wafer or base that forms the bottom of the biological chip plate. In another embodiment, the CCD array is built into the wafer of the biological chip plate.

In another embodiment, the detection device comprises a line scanner, as described in U.S. patent application Ser. No. 08/301,051, filed Sep. 2, 1994, incorporated herein by reference. Excitation optics focuses excitation light to a line at a sample, simultaneously scanning or imaging a strip of the sample. Surface bound labeled targets from the sample fluoresce in response to the light. Collection optics image the emission onto a linear array of light detectors. By employing confocal techniques, substantially only emission from the light's focal plane is imaged. Once a strip has been scanned, the data representing the 1-dimensional image are stored in the memory of a computer. According to one embodiment, a multi-axis translation stage moves the device at a constant velocity to continuously integrate and process data. Alternatively, galvometric scanners or rotating polyhedral mirrors may be employed to scan the excitation light across the sample. As a result, a 2-dimensional image of the sample is obtained.

In another embodiment, collection optics direct the emission to a spectrograph which images an emission spectrum onto a 2-dimensional array of light detectors. By using a spectrograph, a full spectrally resolved image of the sample is obtained.

The read time for a full microtiter plate will depend on the photophysics of the fluorophore (i.e. fluorescence quantum yield and photodestruction yield) as well as the sensitivity of the detector. For fluorescein, sufficient signal-to-noise to read a chip image with a CCD detector can be obtained in about 30 seconds using 3 mW/cm² and 488 nm excitation from an Ar ion laser or lamp. By increasing the laser power, and switching to dyes such as CY3 or CY5 which have lower photodestruction yields and whose emission more closely matches the sensitivity maximum of the CCD detector, one easily is able to read each well in less than 5 seconds. Thus, an entire plate could be examined quantitatively in less than 10 minutes, even if the whole plate has over 4.5 million probes.

A computer can transform the data into another format for presentation. Data analysis can include the steps of determining, e.g., fluorescent intensity as a function of substrate position from the data collected, removing “outliers” (data deviating from a predetermined statistical distribution), and calculating the relative binding affinity of the targets from the remaining data. The resulting data can be displayed as an image with color in each region varying according to the light emission or binding affinity between targets and probes therein.

One application of this system when coupled with the CCD imaging system that speeds performance of the tests is to obtain results of the assay by examining the on or off-rates of the hybridization. In one embodiment of this method, the amount of binding at each address is determined at several time points after the probes are contacted with the sample. The amount of total hybridization can be determined as a function of the kinetics of binding based on the amount of binding at each time point. Thus, it is not necessary to wait for equilibrium to be reached. The dependence of the hybridization rate for different oligonucleotides on temperature, sample agitation, washing conditions (e.g. pH, solvent characteristics, temperature) can easily be determined in order to maximize the conditions for rate and signal-to-noise. Alternative methods are described in Fodor et al., U.S. Pat. No. 5,324,633, incorporated herein by reference.

Assays on biological arrays generally include contacting a probe array with a sample under the selected reaction conditions, optionally washing the well to remove unreacted molecules, and analyzing the biological array for evidence of reaction between target molecules the probes. These steps involve handling fluids. The methods of this invention automate these steps so as to allow multiple assays to be performed concurrently. Accordingly, this invention employs automated fluid handling systems for concurrently performing the assay steps in each of the test wells. Fluid handling allows uniform treatment of samples in the wells. Microtiter robotic and fluid-handling devices are available commercially, for example, from Tecan AG.

The plate is introduced into a holder in the fluid-handling device. This robotic device is programmed to set appropriate reaction conditions, such as temperature, add samples to the test wells, incubate the test samples for an appropriate time, remove unreacted samples, wash the wells, add substrates as appropriate and perform detection assays. The particulars of the reaction conditions depends upon the purpose of the assay. For example, in a sequencing assay involving DNA hybridization, standard hybridization conditions are chosen. However, the assay may involve testing whether a sample contains target molecules that react to a probe under a specified set of reaction conditions. In this case, the reaction conditions are chosen accordingly.

FIG. 3 of Rava et al. depicts an example of a biological chip plate that may be used in the methods of this invention based on the standard 96-well microtiter plate in which the chips are located at the bottom of the wells. Biological chip plates include a plurality of test wells 310, each test well defining an area or space for the introduction of a sample, and each test well comprising a biological chip 320, i.e., a substrate and a surface to which an array of probes is attached, the probes being exposed to the space. FIG. 7 shows a top-down view of a well of a biological chip plate of this invention containing a biological chip on the bottom surface of the well.

This invention contemplates a number of embodiments of the biological chip plate. In a preferred embodiment, depicted in FIG. 4, the biological chip plate includes two parts. One part is a wafer 410 that includes a plurality of biological arrays 420. The other part is the body of the plate 430 that contains channels 440 that form the walls of the well, but that are open at the bottom. The body is attached to the surface of the wafer so as to close one end of the channels, thereby creating wells. The walls of the channels are placed on the wafer so that each surrounds and encloses the probe array of a biological array. FIG. 5 depicts a cross-section of this embodiment, showing the wafer 510 having a substrate 520 (preferably transparent to light) and a surface 530 to which is attached an array of probes 540. A channel wall 550 covers a probe array on the wafer, thereby creating well spaces 560. The wafer can be attached to the body by any attachment means known in the art, for example, gluing (e.g., by ultraviolet-curing epoxy or various sticking tapes), acoustic welding, sealing such as vacuum or suction sealing, or even by relying on the weight of the body on the wafer to resist the flow of fluids between test wells.

In another preferred embodiment, depicted in cross section in FIG. 6, the plates include a body 610 having preformed wells 620, usually flat-bottomed. Individual biological chips 630 are attached to the bottom of the wells so that the surface containing the array of probes 640 is exposed to the well space where the sample is to be placed.

In another embodiment, the biological chip plate has a wafer having a plurality of probe arrays and a material resistant to the flow of a liquid sample that surrounds each probe array. For example, in an embodiment useful for testing aqueous-based samples, the wafer can be scored with waxes, tapes or other hydrophobic materials in the spaces between the arrays, forming cells that act as test wells. The cells thus contain liquid applied to an array by resisting spillage over the barrier and into another cell. If the sample contains a non-aqueous solvent, such as an alcohol, the material is selected to be resistant to corrosion by the solvent.

The microplates of this invention have a plurality of test wells that can be arrayed in a variety of ways. In one embodiment, the plates have the general size and shape of standard-sized microtiter plates having 96 wells arranged in an 8×12 format. One advantage of this format is that instrumentation already exists for handling and reading assays on microtiter plates. Therefore, using such plates in biological chip assays does not involve extensive re-engineering of commercially available fluid handling devices. However, the plates can have other formats as well.

The material from which the body of the biological chip plate is made depends upon the use to which it is to be put. In particular, this invention contemplates a variety of polymers already used for microtiter plates including, for example, (poly)tetrafluoroethylene, (poly)vinylidenedifluoride, polypropylene, polystyrene, polycarbonate, or combinations thereof. When the assay is to be performed by sending an excitation beam through the bottom of the plate collecting data through the bottom of the plate, the body of the plate and the substrate of the chip should be transparent to the wavelengths of light being used.

The arrangement of probe arrays in the wells of a microplate depends on the particular application contemplated. For example, for diagnostic uses involving performing the same test on many samples, every well can have the same array of probes. If several different tests are to be performed on each sample, each row of the plate can have the same array of probes and each column can contain a different array. Samples from a single patient are introduced into the wells of a particular column. Samples from a different patient are introduced into the wells of a different column. In still another embodiment, multiple patient samples are introduced into a single well. If a well indicates a “positive” result for a particular characteristic, the samples from each patient are then rerun, each in a different well, to determine which patient sample gave a positive result.

The biological chip plates used in the methods of this invention include biological chips. The array of probe sequences can be fabricated on the biological chip according to the pioneering techniques disclosed in U.S. Pat. No. 5,143,854, PCT WO 92/10092, PCT WO 90/15070, or U.S. application Ser. Nos. 08/249,188, 07/624,120, and 08/082,937, incorporated herein by reference for all purposes. The combination of photolithographic and fabrication techniques may, for example, enable each probe sequence (“feature”) to occupy a very small area (“site” or “location”) on the support. In some embodiments, this feature site may be as small as a few microns or even a single molecule. For example, a probe array of 0.25 mm² (about the size that would fit in a well of a typical 96-well microtiter plate) could have at least 10, 100, 1000, 10⁴, 10⁵ or 10⁶ features. In an alternative embodiment, such synthesis is performed according to the mechanical techniques disclosed in U.S. Pat. No. 5,384,261, incorporated herein by reference.

Referring to FIG. 8, in general, linker molecules, O-X, are provided on a substrate. The substrate is preferably flat but may take on a variety of alternative surface configurations. For example, the substrate may contain raised or depressed regions on which the probes are located. The substrate and its surface preferably form a rigid support on which the sample can be formed. The substrate and its surface are also chosen to provide appropriate light-absorbing characteristics. For instance, the substrate may be functionalized glass, Si, Ge, GaAs, GaP, SiO2, SiN₄, modified silicon, or any one of a wide variety of gels or polymers such as (poly)tetrafluoroethylene, (Poly)vinylidenedifluoride, polystyrene, polycarbonate, polypropylene, or combinations thereof. Other substrate materials will be readily apparent to those of skill in the art upon review of this disclosure. In a preferred embodiment the substrate is flat glass or silica.

Surfaces on the solid substrate usually, though not always, are composed of the same material as the substrate. Thus, the surface may be composed of any of a wide variety of materials, for example, polymers, plastics, resins, polysaccharides, silica or silica-based materials, carbon, metals, inorganic glasses, membranes, or any of the abovelisted substrate materials. In one embodiment, the surface will be optically transparent and will have surface Si—OH functionalities, such as those found on silica surfaces.

A terminal end of the linker molecules is provided with a reactive functional group protected with a photoremovable protective group, O-X. Using lithographic methods, the photoremovable protective group is exposed to light, hv, through a mask, M₁, that exposes a selected portion of the surface, and removed from the linker molecules in first selected regions. The substrate is then washed or otherwise contacted with a first monomer that reacts with exposed functional groups on the linker molecules (T-X). In the case of nucleic acids, the monomer can be a phosphoramidite activated nucleoside protected at the 5′-hydroxyl with a photolabile protecting group.

A second set of selected regions, thereafter, exposed to light through a mask, M₂, and photoremovable protective group on the linker molecule/protected amino acid or nucleotide is removed at the second set of regions. The substrate is then contacted with a second monomer containing a photoremovable protective group for reaction with exposed functional groups. This process is repeated to selectively apply monomers until polymers of a desired length and desired chemical sequence are obtained. Photolabile groups are then optionally removed and the sequence is, thereafter, optionally capped. Side chain protective groups, if present, are also removed.

The general process of synthesizing probes by removing protective groups by exposure to light, coupling monomer units to the exposed active sites, and capping unreacted sites is referred to herein as “light-directed probe synthesis.” If the probe is an oligonucleotide, the process is referred to as “light-directed oligonucleotide synthesis” and so forth.

The probes can be made of any molecules whose synthesis involves sequential addition of units. This includes polymers composed of a series of attached units and molecules bearing a common skeleton to which various functional groups are added. Polymers useful as probes in this invention include, for example, both linear and cyclic polymers of nucleic acids, polysaccharides, phospholipids, and peptides having either α_-, β_- or ω_-amino acids, heteropolymers in which a known drug is covalently bound to any of the above, polyurethanes, polyesters, polycarbonates, polyureas, polyamides, polyethyleneimines, polyarylene sulfides, polysiloxanes, polyimides, polyacetates, or other polymers which will be apparent upon review of this disclosure. Molecules bearing a common skeleton include benzodiazepines and other small molecules, such as described in U.S. Pat. No. 5,288,514, incorporated herein by reference.

Preferably, probes are arrayed on a chip in addressable rows and columns in which the dimensions of the chip conform to the dimension of the plate test well. Technologies already have been developed to read information from such arrays. The amount of information that can be stored on each plate of chips depends on the lithographic density which is used to synthesize the wafer. For example, if each feature size is about 100 microns on a side, each array can have about 10,000 probe addresses in a 1 cm² area. A plate having 96 wells would contain about 192,000 probes. However, if the arrays have a feature size of 20 microns on a side, each array can have close to 50,000 probes and the plate would have over 4,800,000 probes.

The selection of probes and their organization in an array depends upon the use to which the biological chip will be put. In one embodiment, the chips are used to sequence or re-sequence nucleic acid molecules, or compare their sequence to a referent molecule. Re-sequencing nucleic acid molecules involves determining whether a particular molecule has any deviations from the sequence of reference molecule. For example, in one embodiment, the plates are used to identify in a particular type of HIV in a set of patient samples. Tiling strategies for sequence checking of nucleic acids are described in U.S. patent application Ser. No. 08/284,064 (PCT/US94/12305), incorporated herein by reference.

In typical diagnostic applications, a solution containing one or more targets to be identified (i.e., samples from patients) contacts the probe array. The targets will bind or hybridize with complementary probe sequences. Accordingly, the probes will be selected to have sequences directed to (i.e., having at least some complementarity with) the target sequences to be detected, e.g., human or pathogen sequences. Generally, the targets are tagged with a detectable label. The detectable label can be, for example, a luminescent label, a light scattering label or a radioactive label. Accordingly, locations at which targets hybridize with complimentary probes can be identified by locating the markers. Based on the locations where hybridization occurs, information regarding the target sequences can be extracted. The existence of a mutation may be determined by comparing the target sequence with the wild type.

In a preferred embodiment, the detectable label is a luminescent label. Useful luminescent labels include fluorescent labels, chemi-luminescent labels, bio-luminescent labels, and calorimetric labels, among others. Most preferably, the label is a fluorescent label such as fluorescein, rhodamine, cyanine and so forth. Fluorescent labels include, inter alia, the commercially available fluorescein phosphoramidites such as Fluoreprime (Pharmacia), Fluoredite (Millipore) and FAM (ABI). For example, the entire surface of the substrate is exposed to the activated fluorescent phosphoramidite, which reacts with all of the deprotected 5′-hydroxyl groups. Then the entire substrate is exposed to an alkaline solution (eg., 50% ethylenediamine in ethanol for 1-2 hours at room temperature). This is necessary to remove the protecting groups from the fluorescein tag.

To avoid self-quenching interactions between fluorophores on the surface of a biological chip, the fluorescent tag monomer should be diluted with a non-fluorescent analog of equivalent reactivity. For example, in the case of the fluorescein phosphoramidites noted above, a 1:20 dilution of the reagent with a non-fluorescent phosphoramidite such as the standard 5′-DMT-nucleoside phosphoramidites, has been found to be suitable. Correction for background non-specific binding of the fluorescent reagent and other such effects can be determined by routine testing.

Useful light scattering labels include large colloids, and especially the metal colloids such as those from gold, selenium and titanium oxide.

Radioactive labels include, for example, ³²P. This label can be detected by a phosphoimager. Detection of course, depends on the resolution of the imager. Phosophoimagers are available having resolution of 50 microns. Accordingly, this label is currently useful with chips having features of that size.

The clinical setting requires performing the same test on many patient samples. The automated methods of this invention lend themselves to these uses when the test is one appropriately performed on a biological chip. For example, a DNA array can determine the particular strain of a pathogenic organism based on characteristic DNA sequences of the strain. The advanced techniques based on these assays now can be introduced into the clinic. Fluid samples from several patients are introduced into the test wells of a biological chip plate and the assays are performed concurrently.

In some embodiments, it may be desirable to perform multiple tests on multiple patient samples concurrently. According to such embodiments, rows (or columns) of the microtiter plate will contain probe arrays for diagnosis of a particular disease or trait. For example, one row might contain probe arrays designed for a particular cancer, while other rows contain probe arrays for another cancer. Patient samples are then introduced into respective columns (or rows) of the microtiter plate. For example, one column may be used to introduce samples from patient “one,” another column for patient “two” etc. Accordingly, multiple diagnostic tests may be performed on multiple patients in parallel. In still further embodiments, multiple patient samples are introduced into a single well. In a particular well indicator the presence of a genetic disease or other characteristic, each patient sample is then individually processed to identify which patient exhibits that disease or trait. For relatively rarely occurring characteristics, further order-of-magnitude efficiency may be obtained according to this embodiment.

Particular neurotransmitters receptors were prepared and analyzed in accordance with the invention, and the figures attached hereto are demonstrative of the procedures and results.

Various publications are cited herein including those below, the disclosures of which are incorporated by reference in their entireties.

### REFERENCES

[1] National Household Survey on Drug Abuse: Population Estimates 1996. Substance Abuse and Mental Health Services Administration, Office of Applied Studies. US Department of Health and Human Services, Rockland, Md. July 1997.

[2]Harwood H, Fountain D, Livermore G (1998): The economic costs of alcohol and drug abuse in the United States 1992. NIH (NIDA) Pub Num 98-4327.

[3]Mogil J S, Sternberg W F, Marek P, Sadowski B, Belknap J K, Liebeskind J C (1996): The genetics of pain and pain inhibition. Proc Natl Acad Sci USA 93: 3048-3055.

[4] Pasternak G W (1993): Pharmacological mechanisms of opioid analgesics. Clin Neuropharmacol 16: 1-18.

[5] Grove W M, Eckert E D, Heston L, Bouchard T J J, Segal N, Lykken D T (1990): Heritability of substance abuse and antisocial behavior: a study of monozygotic twins reared apart. Biol Psychiatry 27: 1293-1304.

[6] Kendler K S, Prescott C A, Neale M C, Pedersen N L (1997): Temperance board registration for alcohol abuse in a national sample of Swedish male twins, born 1902 to 1949. Arch Gen Psychiatry 54: 178-184.

[7] Pedersen N L, Floderus-Myrhed B (1984): Twin analysis as a potential tool for examining psychosocial factors associated with and preceding smoking behaviors. Acta Geneticae Medicae Gemellologiae 33: 413-424.

[8] Rounsaville B J, Kosten T R, Weissman M M, Prusoff B, Pauls D, Anton S F, Merikangas K (1991) Psychiatric disorders in relatives of probands with opiate addiction. Arch Gen Psychiatry 48: 33-42.

[9] Tsuang M T, Lyons M J, Eisen S A, Goldberg J, True W, Lin N, Meyer J M, Toomey R, Faraone S V, Eaves L (1996): Genetic influences on DSM-III-R drug abuse and dependence: a study of 3,372 twin pairs. Am J Med Genet 67: 473-477.

[10] Tsuang M T, Lyons M J, Meyer J M, Doyle T, Eisen S A, Goldberg J, True W, Lin N, Toomey R, Eaves L (1998): Co-occurrence of abuse of different drugs in men. Arch Gen Psychiatry 55: 967-972.

[11] Bierut L J, Dinwiddie S H, Begleiter H, Crowe R R, Hesselbrock V, Nurnberger J I, Porjesz B, Schuckit M A, Reich T (1998): Familial transmission of substance dependence: alcohol, marijuana, cocaine, and habitual smoking. Arch Gen Psychiatry 55: 982-988.

[12] Merikangas K R, Stolar M, Stevens D E, Goulet J, Preisig M A, Fenton B, Zhang H, O'Malley S S, Rounsaville B J (1998): Familial transmission of substance use disorders. Arch Gen Psychiatry 55: 973-979.

[13] Sudakov S K, Goldberg S R., Borisova E V, Surkova L A, Turina I V, Rusakov D Ju, Elmer G I (1993): Differences in morphine reinforcement property in two inbred rat strains: associations with cortical receptors, behavioral activity, analgesia and the cataleptic effects of morphine. Psychopharmacol 112: 183-188.

[14] Schlussman S D, Ho A, Zhou Y, Curtis A E, Kreek M J (1998): Effects of “binge” pattern cocaine on stereotypy and locomotor activity in C57BL/6J and 129/J mice. Pharmacol Biochem Behav 60: 593-599.

[15] Elmer G I, Mathura C B, Goldberg S R (1993): Genetic factors in conditioned tolerance to the analgesic effects of etonitazene. Pharmcol Biochem Behav 45: 251-253.

[16] Elmer G I, Pieper J O, Goldberg S R, George F R (1995): Opioid operant self-administration, analgesia, stimulation and respiratory depression in mu-deficient mice. Psychopharmacology 117: 23-31.

[17] Elmer G I, Pieper J O, Negus S S, Woods J H (1998): Genetic variance in nociception and its relationship to the potency of morphine-induced analgesia in thermal and chemical tests. Pain 75: 129-140.

[18] Mansson E, Bare L, Yang D (1994): Isolation of a human k-opioid receptor cDNA from placenta. Biochem Biophys Res Commun 202: 1431-1437.

[19] Simonin F, Gaveriaux-Ruff C, Befort K, Matthes H, Lannes B, Micheletti G, Mattei M-G, Charron G, Bloch B, Kieffer, B (1995): k-Opioid receptor in humans: cDNA and genomic cloning, chromosomal assignment, functional expression, pharmacology, and expression pattern in the central nervous system. Proc Natl Acad Sci USA 92: 7006-7010.

[20] Horikawa S, Takai T, Toyosato M, Takahashi H, Noda M, Kakidani H, Kubo T, Hirose T, Inayama S, Hayashida H, Miyata T, Numa S (1983): Isolation and structural organization of the human preproenkephalin B gene. Nature 306: 611-614.

[21] Mollereau C, Parmenteir M, Mailieux P, Butour J-L, Moisand C, Chalon P, Caput D, Vassart G, Meunier J-C (1994): ORL1, a novel member of the opioid receptor family. Cloning, functional expression and localization. FEBS Lett 341: 33-38.

[22] Mollereau C, Simons M-J, Soularue P, Liners F, Vassart G, Meunier,J-C, Parmentier M (1996): Structure, tissue distribution, and chromosomal localization of the prepronociceptin gene. Proc Natl Acad Sci USA 93: 8666-8670.

[23] Noda M, Teranishi Y, Takahashi H, Toyosato M, Notake M, Nakanishi S, Numa S (1982): Isolation and structural organization of the human preproenkephalin gene. Nature 297: 431-434.

[24] Comb M, Seeburg P H, Adelman J, Eiden L, Herbert E (1982): Primary structure of the human Met- and Leu-enkephalin precursor and its mRNA. Nature, 295: 663-666.

[25] Shibahara S, Morimoto Y, Furutani Y, Notake M, Takahashi H, Shimizu S, Horikawa S, Numa S (1983): Isolation and sequence analysis of the human corticotropin-releasing factor precursor gene. EMBO j., 2: 775-779.

[26] Chen R, Lewis K A, Perrin M H, Vale W W (1993): Expression cloning of a human corticotropin-releasing-factor receptor. Proc Natl Acad Sci USA 90: 8967-8971.

[27] Vita N, Laurent P, Lefort S, Chalon P, Lelias J M, Kaghad M, Le Fur G, Caput D, Ferrara P (1993): Primary structure and functional expression of mouse pituitary and human brain corticotrophin releasing factor receptors. FEBS Lett 335: 1-5.

[28] Chang A C, Cochet M, Cohen S N (1980): Structural organization of human genomic DNA encoding the pro-opiomelanocortin peptide. Proc Natl Acad Sci USA 77: 4890-4894.

[29] Whitfeld P L, Seeburg P H, Shine J (1982): The human pro-opiomelanocortin gene: organization, sequence, and interspersion with repetitive DNA. DNA 1: 133-143.

[30] Takahashi H, Teranishi Y, Nakanishi S, Numa S (1981): Isolation and structural organization of the human corticotropin--lipotropin precursor gene. FEBS Lett 135: 97-102, 1981.

[31] Yuferov V, Zhou Y, Spangler R, Maggos C E, Ho A, Kreek M J (1999): Acute “binge” cocaine increases mu opioid receptor mRNA levels in areas of the rat mesolimbic mesocortical dopamine system. Brain Res Bulletin in press.

[32] Unterwald E M, Horne-King J, Kreek, M J (1992): Chronic cocaine alters brain mu opioid receptors. Brain Res 584: 314-318.

[33] Hand T H, Koob G F, Stinus L, LeMoat M (1988): Aversive properties of opiate receptor blockade are centrally mediated and are potentiated by previous exposure to opiates. Brain Res 474: 364-368.

[34] Kreek M J, Hartman N (1982): Chronic use of opioids and antipsychotic drugs: side effects, effects on endogenous opioids and toxicity. Ann NY Acad Sci 398: 151-172.

[35] Kosten T R, Kreek M J, Raghunath J, Kleber H D (1986): Cortisol levels during chronic naltrexone maintenance treatment in ex-opiate addicts. Biological Psychiatry 21: 217-220.

[36] Kosten T R, Kreek M J, Raghunath J, Kleber H D (1986): A preliminary study of beta-endorphin during chronic naltrexone maintenance treatment in ex-opiate addicts. Life Sciences 39: 55-59.

[37] Volpicelli J R, Alterman Al, Hayashida M, O'Brien C P (1992): Naltrexone in the treatment of alcohol dependence. Arch Gen Psychiatry 49: 876-880.

[38] Volpicelli J R, Watson N T, King A C, Sherman C E, O'Brien CP (1995): Effect of naltrexone on alcohol “high” in alcoholics. Amer J of Psych 152: 613-15.

[39] O'Malley S S, Jaffe A J, Change G, Schottenfeld R S, Meyer R E, Rounsaville B J (1992): Naltrexone and coping skills therapy for alcohol dependence. Arch Gen Psychiatry 49: 881-887.

[40] Mason B J, Ritvo E C, Morgan R O, Salvato F R, Goldberg G, Welch B, Mantero-Atienza E (1994): A double-blind, placebo-controlled pilot study to evaluate the efficacy and safety of oral nalmefene HCl for alcohol dependence. Alcohol Clin Exp Res 18: 1162-1167.

[41] King A C, Volpicelli J R, Gunduz M, O'Brien C O, Kreek M J (1997): Naltrexone biotransformation and incidence of subjective side effects: a preliminary study. Alcoholism Clin and Exp Res 21(5): 906-909.

[42] Kreek M J (1981): Metabolic interactions between opiates and alcohol. Ann NY Acad Sci 362: 36-49.

[43] Adams J U, Holtzman S G (1990): Pharmacologic characterization of the sensitization to the rate-decreasing effects of naltrexone induced by acute opioid pretreatment in rats. J Pharmacol Exp Ther 253: 483-489.

[44] Bickel W K, Stitzer M L, Licbson I A, Bicclow O E (1988): Acute physical dependence in man: effects of naloxone after brief morphine exposure. J Pharmacol Exp Ther 244: 126-132.

[45] Culpepper-Morgan J A, Kreek M J (1997): HPA axis hypersensitivity to naloxone in opioid dependence: a case of naloxone induced withdrawal. Metabolism 46: 130-134.

[46] Culpepper-Morgan J A, Inturrisi C E, Portenoy R K, Foley K, Houde R W, Marsh F, Kreek M J (1992): Treatment of opioid induced constipation with oral naloxone: a pilot study. Clin Pharm Ther 23: 90-95.

[47] Heishman S J, Stitzer N I L, Bigelow G E, Liebson I A (1939): Acute opioid physical dependence in humans: effect of varying the morphine-naloxone interval. J Pharmacol Exp Ther 250: 485-491.

[48] Heishman S J, Stitzer N I L, Bigelow G E, Liebson I A (1989): Acute opioid physical dependence in post-addict humans: naloxone dose effects after brief morphine exposure. J Pharmacol Exp Ther 248: 127-134.

[49] Jones R T (1979): Dependence in non-addict humans after a single dose of morphine. In: Way E L ed Endogenous and Exogenous Opioid Agonists and Antagonists. Pergamon Press, New York.

[50] Rosen M I, McMahon T J, Margolin A, Gill T S, Woods S W, Pearsall H R, Kreek M J, Kosten T R (1995): Reliability of sequential naloxone challenge tests. Amer J Drug Alc Abuse 4: 453-467.

[51] Rosen M I, McMahon T J, Hameedi F A, Pearsall H R, Woods S W, Kreek M J, Kosten T R (1996): Effect of clonidine pretreatment on naloxone-precipitated opiate withdrawal. J Pharmacol Exp Ther 276: 1128-1135.

[52] Berrettini W H, Ferraro T N, Alexander A C, Buchberg A M, Vogel W H (1994): Quantitative trait loci mapping of three loci controlling morphine preference using inbred mouse strains. Nature Genet 7: 54-58.

[53] Alexander R C, Heydt D, Ferraro T N, Vogel W, Berrettini W H (1996): Further evidence for a quantitative trait locus on murine chromosome 10 controlling morphine preference in inbred mice. Psychiatr Genet 6: 29-31.

[54] Kozak C A, Fillie J, Adamson M C, Chen Y, Yu L (1994): Murine chromosomal location of the mu and kappa opioid receptor genes. Genomics 21: 659-661.

[55] Bergen A W, Kokoszka J, Peterson R, Long J C, Virkkunen M, Linnoila M, Goldman D (1997): Mu opioid receptor gene variants: lack of associaton with alcohol dependence. Mol Psychiatry 2: 490-494.

[56] Berrettini W H, Hoehe M R, Ferrada T N, Gottheil E (1997): Human mu opioid receptor gene polymorphisms and vulnerability to substance abuse. Addiction Biol 2: 303-308.

[57] Bond C, LaForge K S, Tian M, Melia D, Zhang S, Borg L, Gong J, Schluger J, Strong J A, Leal S M, Tischfield J A, Kreek M J, Yu L (1998): Variation in receptor function from a single nucleotide polymorphism in the human mu opioid receptor gene: possible implications for opioid addiction. Proc Nat Acad Sci (USA) 95: 9608-9613.

[58] Spangler R, Ho A, Zhou Y, Maggos C, Yuferov V, Kreek, M J (1996): Regulation of kappa opioid receptor mRNA in the rat brain by “binge” pattern cocaine administration and correlation with preprodynorphin mRNA. Mol Brain Res 38: 71-76.

[59] Spangler R, Zhou Y, Maggos C E, Schlussman S, Ho A, Kreek M J (1997): Persistent preprodynorphin and kappa opioid receptor mRNA responses to cocaine occur acutely. Abstracts of the 59th Annual Scientific Meeting of the College on Problems of Drug Dependence, Nashville, Tenn., June.

[60] Spangler R, Zhou Y, Maggos C E, Schlussman S D, Ho A, Kreek, M J (1997): Prodynorphin, proenkephalin and kappa opioid receptor mRNA responses to acute “binge” cocaine. Mol Brain Res 44: 139-142.

[61] Unterwald E M, Kreek M J (1993): Kappa and delta opioid receptor densities following chronic repeated cocaine administration. In: Abstracts of the International Narcotics Research Conference.

[62] Hurd Y L, Brown E E, Finlay J M, Fibiger H C, Gerfen, C R (1992): Cocaine self-administration differentially alters mRNA expression of striatal peptides. Mol Brain Res 13: 165-170.

[63] Daunais J B, Roberts D C, McGinty J F (1993): Cocaine self-administration increases preprodynorphin, but not c-fox, mRNA in rat striatum. NeuroReport 4: 543-546.

[64] Kreek M J, Schluger J, Borg L, Gunduz M, Ho, A (1999): Dynorphin A1-13 causes elevation of serum levels of prolactin through an opioid receptor mechanism in humans: gender differences and implications for modulation of dopaminergic tone in the treatment of addictions. J Pharmacol Exp Ther 288: 260-269.

[65] Yasuda K, Espinos III R, Takeda J, Le Beau M M, Bell G I (1994): Localization of the kappa opioid receptor gene to human chromosome band 8q11.2. Genomics 19: 596-597.

[66] Litt M, Buroker N E, Kondoleon S, Douglass J, Liston D, Sheehy R, Magenis R E (1988): Chromosomal localization of the human proenkephalin and prodynorphin genes. Am J Hum Genet 42: 327-334.

[67] Geijer T, Jonsson E, Neiman J, Gyllander A, Sedvall F, Rydberg, U, Terenius L (1997): Prodynorphin allelic distribution in Scandinavian chronic alcoholics. Alcohol Clin Exper Res 21: 1333-1336.

[68] Lightman S L, Young W S, III (1987): Changes in hypothalamic preproenkephalin A mRNA following stress and opiate withdrawal. Nature 328: 643-645.

[69] Uhl G R, Ryan J P, Schwartz J P (1988): Morphine alters preproenkephalin gene expression. Brain Res 459: 391-397.

[70] Cohen B M, Van Nguyen T, Hyman S E (1990): Cocaine induced changes in the rat brain. NIDA Res Monograph 105: 175-181.

[71] Borsook D, Falkowski 0, Rosen H, Comb M, Hyman S E (1994): Opioids modulate stress-induced proenkephalin gene expression in the hypothalamus of transgenic mice: a model of endogenous opioid gene regulation by exogenous opioids. J Neurosci 14: 7261-7271.

[72] LaForge K S, Yuferov V, Zhou Y, Ho A, Kreek M J (1998): “Binge” cocaine administration alters preproenkephalin mRNA levels in the guinea pig brain. In Harris L S ed Problems of Drug Dependence, 1997; Proceedings of the 59th Annual Scientific Meeting of the College on Problems of Drug Dependence. National Institute of Drug Abuse Research Monograph Series Washington, D.C. Supt of Docs US Govt Print Off DHHS Pub No (ADM)98-4305 178: 83.

[73] Hurd Y L, Herkenham M (1993): Molecular alterations in the neostriatum of human cocaine addicts. Synapse 13: 357-369.

[74] Chan R J, McBride A W, Thomasson H R, Ykenney A, Crabb D W (1994): Allele frequencies of the preproenkephalin A (PENK) gene CA repeat in Asians, African-Americans, and Caucasians: lack of evidence for different allele frequencies in alcoholics. Alcohol Clin Exp Res 18: 533-535.

[75] Mikesell M J, Sobell J L, Sommer S S, McMurray C T (1996): Identification of a missense mutation and several polymorphisms in the proenkephalin A gene of schizophrenic patients. Am J Med Genet 67: 459-467.

[76] Mikesell M J, Barron Y D, Nimgaonkar V L, Sobell J L, Sommer S S, McMurray C T (1997): Gly(247)aAsp proenkephalin A mutation is rare in schizophrenia populations. Am J Med Genet 74: 213-215.

[77] Darland T, Heinricher M M, Grandy D K (1998): Orphanin FQ/nociceptin: a role in pain and analgesia, but so much more. Trends in Neurosci 21(5): 215-221.

[78] Meunier J-C, Mollereau C, Toll L, Suaudeau C, Moisand C, Alvinerie P, Butour J-L, Guillemot J-C, Ferrara P, Monsarrat B, Mazargull H, Vassart G, Parmentier M, Costentin J (1995): Isolation and structure of the endogenous agonist of opioid receptor-like ORL1 receptor. Nature 377: 532-535.

[79] Lightman S L, Young W S, III (1988): Corticotropin-releasing factor, vasopressin and pro-opiomelanocortin mRNA responses to stress and opiates in the rat. J Physiol 403: 511-523.

[80] Mocchetti I, Ritter A, Costa E (1989): Down-regulation of proopiomelanocortin synthesis and beta-endorphin utilization in hypothalamus of morphine-tolerant rats. J Mol Neurosci 1: 33-38.

[81] Bronstein D M, Przewlocki R, Akil H (1990): Effects of morphine treatment on proopiomelanocortin systems in rat brain. Brain Res 519: 102-111.

[82] Gudehithlu de Yebenes E, Tejwani G A, Bhargava H N (1991): Beta-endorphin and methionine-enkephalin levels in discrete brain regions, spinal cord, pituitary gland and plasma of morphine tolerant-dependent and abstinent rats. Brain Res 553: 284-290.

[83] Wardlaw S L, Kim J & Sobieszczyk S 1996 Effect of morphine on proopiomelanocortin gene expression and peptide levels in the hypothalamus. Mol Brain Res 41: 140-147.

[84] Kreek M J (1973): Medical safety and side effects of methadone in tolerant individuals. J Amer Med Assn 223: 665-668.

[85] Kreek M J (1978): Medical complications in methadone patients. Ann NY Acad Sci 311: 110-134.

[86] Kreek M J, Wardlaw S L, Friedman J, Schneider B, Frantz AG (1981): Effects of chronic exogenous opioid administration on levels of one endogenous opioid (beta-endorphin) in man. Advances in Endogenous and Exogenous Opioids, Simon E, Takagi H eds Tokyo, Japan: Kodansha Ltd. Publishers, 364--366.

[87] Kreek M J, Raghunath J, Plevy S, Hamer D, Schneider B, Hartman N (1984): ACTH, cortisol and beta-endorphin response to metyrapone testing during chronic methadone maintenance treatment in humans. Neuropeptides, 5: 277-278.

[88] Kreek M J (1992): Rationale for maintenance pharmacotherapy of opiate dependence. In: C P O'Brien, J H Jaffe eds Addictive States. Raven Press, Ltd.:New York, 205-230.

[89] Kreek M J (1996): Opiates, opioids and addiction. Mol Psych 1: 232-254.

[90] Kreek M J (1996): Opioid receptors: Some perspectives from early studies of their role in normal physiology, stress responsivity and in specific addictive diseases. Journal of Neurochemical Res 21: 1469-1488.

[931] Rivier C, Vale W (1987): Cocaine stimulates adrenocorticotropin (ACTH) secretion through a corticotropin-releasing factor (CRF)-mediated mechanism. Brain Res 422: 403-406.

[92] Rivier C, Lee S (1994): Stimulatory effect of cocaine on ACTH secretion: Role of the hypothalamus. Mol Cell Neurosci 5: 189-195.

[93] Zhou Y, Spangler R, LaForge K S, Maggos C E, Ho A, Kreek M J (1996): Corticotropin-releasing factor and CRF-R1 mRNAs in rat brain and pituitary during “binge” pattern cocaine administration and chronic withdrawal. J Pharmacol Exp Ther 279: 351-358.

[94] Sarnyai Z, Biro E, Gardi J, Vecsernyes M, Julesz J, Telegdy G (1995): Brain corticotropin-releasing factor mediates ‘anxiety-like’ behavior induced by cocaine withdrawal in rats. Brain Res 675: 89-97

[95] DeVries A C, Pert, A (1998): Conditioned increases in anxiogenic-like behavior following exposure to contextual stimuli associated with cocaine are mediated by corticotropin-releasing factor. Psychopharmacology 137(4): 333-40.

[96] Ambrosio E, Sharpe L G, Pilotte N S (1997): Regional binding to corticotropin releasing factor receptors in brain of rats exposed to chronic cocaine and cocaine withdrawal. Synapse 25(3): 272-6.

[97] Zhou Y, Spangler R, LaForge K S, Maggos C E, Kreek, M J (1996): Modulation of CRF-mRNA in rat anterior pituitary by dexamethasone: correlation with POMC mRNA. Peptides 17: 435-441.

[98] Richter R M, Pich E M, Koob G F, Weiss F (1995): Sensitization of cocaine-stimulated increase in extracellular levels of corticotropin-releasing factor from the rat amygdala after repeated administration as determined by intracranial microdialysis. Neuroscience Letters 187(3): 169-72.

[99] Sarnyai Z, Biro E, Penke B, Telegdy G (1992): The cocaine-induced elevation of plasma corticosterone is mediated by endogenous corticotropin-releasing factor (CRF) in rats. Brain Res 589(1): 154-6.

[100] Goeders N E, Bienvenu O J, De Souza E B (1990): Chronic cocaine administration alters corticotropin-releasing factor receptors in the rat brain. Brain Res 531(1-2): 322-8.

[101] Schluger J H, Perret G, McClary K, Gunduz M, Disla I, Myers J E, Daniels J, Ho A, Kreek, M J (1998): Differential stimulation of the HPA axis by CRF in normal volunteers and in patients with addictions. In: Harris LS ed Problems of Drug Dependence 1998; Proceedings of the 60th Annual Scientific Meeting for the College on problems of Drug Dependence. National Institute of Drug Abuse Research Monograph Series, Washington D.C. Supt of Docs US Govt Print Off, in press.

[102] Knych E T, Prohaska J R (1981): Effect of chronic intoxication and naloxone on the ethanol-induced increase in plasma corticosterone, Life Sci 28: 1987-1994

[103] Guaza C, Torrellas A, Borrell S (1983): Adrenocortical response to acute and chronic ethanol administration in rats, Psychopharmacology 79: 173-176.

[104] Rivier C, Vale W (1988): Interaction between ethanol and stress on ACTH and beta-endorphin secretion, Alcohol Clin Exp Res, 12: 206-210.

[105] Rivier C, Imaki T, Vale W (1990): Prologed exposure to alcohol: effect on CRF mRNA levels, and CRF- and stress-induced ACTH secretion in the rat, Brain Res 520: 1-5.

[106] Arbiser J L, Morton C C, Bruns G A P, Majzoub J A (1988): Human corticotropin releasing hormone gene is located on the long arm of chromosome 8. Cell Genet 47: 113-116.

[107] Vamvakopoulos N C, Chrousos G P (1993): Regulated activity of the distal promoter-like element of the human corticotropin-releasing hormone gene and secondary structural features of its corresponding transcripts. Mol Cell Endocrinol 94: 73-78.

[108] Baerwald C G, Panayai G S, Lanchbury J S (1997): A new Xmnl polymorphism in the regulatory region of the corticotropin releasing hormone gene. Hum Genet 97: 697-698.

[109] Baerwald C G, Panayai G S, Lanchbury J S (1997): Corticotropin releasing hormone promoter region polymorphisms in rheumatoid arthritis. J Rheumatol 24: 215-216.

[110] Gu J, Sadler L, Daiger S, Wells D, Wagner M (1993): Dinucleotide repeat polymorphism at the CRH gene. Hum Mol Genet 2: 85.

[111] Stratakis C A, Sarlis N J, Berrettini W H, Badner J A, Chrousos G P, Gershon E S, Detera-Wadleigh S D (1997): Lack of linkage between the corticotropin-releasing hormone (CRH) gene and bipolar affective disorder. Mol Psychiatry 2: 483-485.

[112] Kyllo J H, Collins M M, Vetter K L, Cuttler L, Rosenfield R L, Donohoue P A (1996): Linkage of congenital isolated adrenocorticotropic hormone deficiency to the corticotropin releasing hormone locus using simple sequence repeat polymorphisms. Am J Med Genet 62: 262-267.

[113] Vamvakopoulos N C, Sioutopoulou T O (1994): Human corticotropin-releasing hormone receptor gene (CRHR) is located on the long arm of chromosome 17 (17q12-qter). Chromosome Res 2: 471-473.

[114] Polymeropoulos M H, Torres R, Yanovski J A, Chandrasekharappa S C, Ledbetter D H (1995): The human corticotropin-releasing factor receptor (CRHR) gene maps to chromosome 17q12-q22. Genomics 28: 123-124.

[115] Hughes J, Smith T W, Kosterlitz H W, Fothergill L A, Morgan M A, Morris H R (1975): Identification of two related pentapeptides from the brain with potent opiate agonist activity. Nature 258: 577-580.

[116] Przewlocki R, Hollt V, Duka T H, Kleber G, Gramsch C H, Haarmann I, Herz A (1979): Long-term morphine treatment decreases endorphin levels in rat brain and pituitary. Brain Res 174: 357-361.

[117] Ragavan W, Wardlaw S L, Kreek M J, Frantz A G (1983): Effects of chronic naltrexone and methadone administration on brain immunoreactivity beta-endorphin in the rat. Neuroendocrinology 37: 266-268.

[118] Schluger J, Bodner G, Gunduz M, Ho A, Kreek, M J (1998): Abnormal metyrapone tests during cocaine abstinence. In: Harris L S ed Problems of Drug Dependence 1997; Proceedings of the 59th Annual Scientific Meeting of the College on Problems of Drug Dependence. National Institute of Drug Abuse Research Monograph Series, Washington, D.C.: Supt of Docs, US Govt Print Off DHHS Pub No (ADM)98-4305,178: 105.

[119] Aouizerate B, Schluger J H, Perret G, McClary K, Ho A, Piazza P V, Kreek, M J (1999): Enhanced sensitivity to negative glucocorticoid feedback in methadone patients with ongoing cocaine dependence. In: Harris L S (ed.), Problems of Drug Dependence, 1998; Proceedings of the 60th Annual Scientific Meeting of the College on problems of Drug Dependence. National Institute of Drug Abuse Research Monograph Series, Washington, D.C.: Supt Of Docs, US Govt Print Off, in press.

[120] Seizinger B R, Bovermann K, Holit V, Herz A (1984): Enhanced activity of the endorphinergic system in the anterior and neurointermediate lobe of the rat pituitary gland after chronic treatment with ethanol liquid diet. J Pharmacol Exp Ther 230: 455-461.

[121] Gianoulakis C, Hutchison W D, Kalant H (1988): Effects of ethanol treatment and withdrawal on biosynthesis and processing of proopiomelanocortin by the rat neurointermediate lobe. Endocrinology 122: 817-825.

[122] Dave J R, Eiden L E, Karanian J W, Eskay R L (1986): Ethanol exposure decreases pituitary corticotropin-releasing factor binding, adenylate cyclase activity, proopiomelanocortin biosynthesis and plasma beta-endorphin levels in the rat. Endocrinology 118: 280-286.

[123] Angelogianni P, Gianoulakis C (1993): Chronic ethanol increases proopiomelanocortin gene expression in the rat hypothalamus. Neuroendocrinology 57: 106-114.

[124] Kreek M J, Koob G F (1998): Drug dependence: Stress and dysregulation of brain reward pathways. Drug and Alc Dep 51: 23-47.

[125] Owerbach D, Rutter W J, Roberts J L, Wittfield P, Shine J, Seeburg P H, Shows T B (1981): The proopiocortin (adrenocorticotropin/b-lipoprotein) gene is located on chromosome 2 in humans. Somatic Cell Genet 7: 359-369.

[126] Zabel B U, Naylor S L, Sakaguchi A Y, Bell G I, Shows T B (1983): High-resolution chromosomal localization of human genes for amylase, proopiomelanocortin, somatostatin, and a DNA fragment (D3S1) by in situ hybridization. Proc Natl Acad Sci USA 80: 6932-6936.

[127] Feder J, Migone N, Chandg A C, Cochet M, Cohen S N, Cann H, Cavalli-Sforza L L (1983): A DNA polymorphism in close physical linkage with the proopiomelanocortin gene. Am J Hum Genet 35: 1090-1096.

[128] Feder J, Gurling H M, Darby J, Cavalli-Sforza L L (1985): DNA restriction fragment analysis of the proopiomelanocortin gene in schizophrenia and bipolar disorders. Am J Hum Genet 37: 286-294.

[129] Gostout B, Liu Q, Sommer S S (1993): “Cryptic” repeating triplets of purines and pyrimidines (cRRY(i)) are frequent and polymorphic: analysis of coding cRRY (I) in the proopiomelanocortin (POMC) and TATA-binding protein (TPB) genes. Am J Hum Genet 52: 1182-1190.

[130] Morris J C, Bertram C E, Lowry P J, Savva D (1994): Cryptic trinucleotide repeat polymorphism in the POMC gene. Hum Mol Genet 3: 2080.

[131] Krude H, Biebermann H, Luck W, Horn R, Brabant G, Gruters A (1998): Severe early-onset obesity, adrenal insufficiency and red hair pigmentation caused by POMC mutations in humans. Nature Genetics 19: 155-157.

[132] Befort K, Mattei M G, Roeckel N, Kieffer, B L (1994): Chromosomal localization of the d opioid receptor gene to human Ip34.3-p36.1 and mouse 4D bands by in situ hybridization. Genomics 20: 143-145.

[133] Mayer P, Rochlitz H, Rauch E, Rommelspacher H, Hasse H E, Schmidt S, Höllt V (1997): Association between a delta opioid receptor gen polymorphism and heroin dependence in man. NeuroReport 8: 2547-2550.

[134] Orita M, Suzuki Y, Sekiya T and Hayashi K (1989): Rapid and sensitive detection of point mutations and DNA polymorphisms using the polymerase chain reaction. Genomics 5: 874-879.

[135] Hayashi K (1991): PCR-SSCP: a simple and sensitive method for detection of mutations in the genomic DNA. PCR Methods Appl 1: 34-38.

[136] White M B, Carvalho M, Derse D, O'Brian S J, and Dean M (1993): Detecting single base substitutions as heteroduplex polymorphisms. Genomics 12: 3031-306.

[137] Grompe M (1993): The rapid detection of unknown mutations in nucleic acids. Nature Genet 5: 111-117.

[138] Wu D Y, Ugozzoli L, Pal B K, Wallace R B (1989): Allele-specific enzymatic amplification of beta-globin genomic DNA for diagnosis of sickle cell anemia. Proc Natl Acad Sci USA 86: 2757-2760.

[139] Sarkar G, Cassady J, Bottema C D K, and Sommer, S S (1990): Characterization of polymerase chain reaction amplification of specific alleles. Anal Biochem 186: 64-68.

[140] Hayashi K, Yandell D W (1993): How sensitive is PCR-SSCP? Hum Mutat 2: 338-346.

[141] Jordanova A, Kalaydjieva L, Savov A, Claustress M, Schwartz M, Estivill X, Angeligheva D, Haworth A Casals T, Krememsky I (1997): SSCP analysis: A blind sensitivity trial. Hum Mutat 10: 65-70.

[142] Lander E S (1999): Array of hope. Nature Genetics suppl 21: 3-5.

[143] Southern E, Kalim M, Shchepinov M (1999): Molecular interactions on microarrays. Nature Genetics suppl 21: 5-9.

[144] Cheung V G, Morley M, Aguilar F, Massimi A, Kucherlapti F, Childs G (1999): Making and reading microarrays. Nature Genetics suppl 21: 15-19.

[145] Lipshutz, R J, Fodor P A, Gingeras T R, Lockhart D J (1999): High density synthetic oligonucleotide arrays. Nature Genetics suppl 21: 20-24.

[146] Hacia J G (1999): Resequencing and mutational analysis using oligonucleotide microarrays. Nature

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, given for nucleic acids or polypeptides are approximate, and are provided for description.

What is claimed is:

1. A method for making a biological chip plate comprising the steps of:
(a) providing a body comprising a plurality of wells defining spaces;
(b) providing a wafer comprising on its surface a plurality of probe arrays, each probe array comprising a collection of probes, at least two of which are different, arranged in a spacially defined and physically addressable manner;
(c) attaching the wafer to the body so that the probe arrays are exposed to the spaces of the wells;
(d) wherein the probe arrays are selected from a family of neurotransmitter genes known to be affected by exposure to addictive agents and/or alcohol

2. The method of claim 1 wherein the probes are DNA and/or RNA molecules.

3. A method for making a biological chip plate comprising the steps of providing a wafer comprising on its surface a plurality of probe arrays, each probe array comprising a collection of probes, at least two of which are different, arranged in a spacially defined and physically addressable manner; and applying a material resistant to the flow of a liquid sample so as to surround the probe arrays, thereby creating test wells.

4. The method of claim 3 wherein the probes are DNA or RNA molecules.
